(19) United States
(12) Patent Application Publication
(10) Pub. No.: US 2010/0030081 A1
(43) Pub. Date: Feb. 4, 2010

(54) ULTRASONOGRAPH

(76) Inventors: Hiroshi Masuzawa, Machida (JP); Satoshi Tamano, Kashiwa (JP); Mitsuhiro Oshiki, Tokyo (JP); Hiroshi Kuribara, Abiko (JP)

Correspondence Address: ANTONELLI, TERRY, STOUT & KRAUS, LLP, 1300 NORTH SEVENTEENTH STREET, SUITE 1800, ARLINGTON, VA, 22209-3873, US

(21) Appl. No.: 12/529,038

(22) PCT Filed: Jan. 23, 2008
(86) PCT No.: PCT/JP2008/050862

§ 371 Date: Aug. 28, 2009

(30) Foreign Application Priority Data
Mar. 6, 2007 (JP) 2007-056282

(51) Int. Cl. A61B 8/14

(52) U.S. Cl. 600/459

(57) ABSTRACT

Multidimensional beamformer data including time, which is provided by numerical data in practical use, is efficiently stored in storage means and is computed. The computation means for the beamformer utilizes beamformer data computing means which performs the computation of delays and weighting by using a linear combination function of a 2nd-degree term of time and n number of any parameters P₁ to Pₙ, a 2nd-degree term of time, a 1st-degree term of P₁ to Pₙ, and a constant term, thereby achieving high storage efficiency and enabling time-wise continuous adjustment of parameters.

### TECHNICAL FIELD

The present invention relates to a diagnostic ultrasound imaging system equipped with a beamformer which enables high accuracy scanning.

### BACKGROUND ART

In a generating method of a transmitting time delay and receiving time delay in a conventional medical ultrasound imaging system, a method of computing and generating delay times in real time within the equipment is known. In such techniques, there is known a method in which successive computation is performed by finding a recurrence equation relation, which is successively updated from the functional relationship of sound wave propagation time, assuming a sound velocity based on a distance relational expression among a reference point of spatial position of a receiving transducer array element of a probe, a transmitting focal point position and a time-varying receiving focal point position, and spatial positions of the elements within a body of patients under examination. For example, in U.S. Pat. No. 5,522,391, regarding the sound velocity Vc, a range (distance) R between a focus and the center reference of an array, a range r between the focus and the i-th transmitter/receiver element, and a delay time τᵢ to be added; from the relationship

$$\frac{R}{Vc} + \frac{{r}_{i}}{Vc} + {τ}_{i} = \frac{2 R}{Vc} \qquad [ Expression 1 ]$$

an approximate computation to a desired accuracy is realized by determining a recurrence equation by dividing the range R into piecewise ones. Further, in U.S. Pat. No. 4,949,259, a function for providing a relational expression of delay time determined by a similar relationship as described above is computed by an accumulator by dividing it into multiple regional sections depending on the range R based on the Maclaurin expansion equation.
Patent Document 1: U.S. Pat. No. 5,522,391
Patent Document 2: U.S. Pat. No. 4,949,259

### DISCLOSURE OF THE INVENTION

### Problems to be Solved by the Invention

Compared with the time when the above described techniques were disclosed, since the integrated circuit technologies have made a dramatic progress, computations to realize more complicated features have become required. In dynamic receive focusing in which focuses of received signals are updated successively in range direction, the delay time is optimized by a complicated numerical calculation especially in a near-field of sound field, there is a case in which setting of a delay time change which cannot be expressed by the analytic functional relationships disclosed in the above described documents. Those are usually set at the same time not only with the delay time but with aperture amplitude weighting (apodization). Hereafter, delay time data in conjunction with aperture amplitude weighting data, which are set as parameters for arbitrary sign coefficients, are referred to as “the beamformer data”. The beamformer data are set for every channel and beamforming condition.

For example, there may be a case of realizing a request not to arrange the resolution of the azimuthal direction of the beam to conform to the diffraction beamwidth determined by the aperture size and range, but to arrange the beamwidth of main lobe of the beam to be constant within a target range to suppress the variation of the resolution of pulse of transmit and receive (interrogating pulse); or a case in which it is required to realize operation of beamformer in such a manner as to suppress sensitivity of suppressed sidelobe response for the orientation other than an intended direction below a predetermined level by distributing the response sensitivity in accordance with demand. In those cases, so as to meet the predetermined regularizing condition (constraint condition) of a directivity function, the beamformer data may be determined, for example, from a large number of results of optimal condition finding by linear programming. When performing the real-time creation of beamformer data in accordance with an optimal condition, a solving solution by an analytic function representation such as those described in U.S. Pat. No. 5,522,391 and U.S. Pat. No. 4,949,259 requires the setting of a large number of constraints, and therefore the amount of computation increases making it difficult to be realized.

Further, in the latest beamformer technologies, in order to realize a high accuracy, a received signal is converted into a digital signal by using an analog-to-digital converter (hereafter, referred to as “ADC”) and a numerical computation is utilized to realize the purpose. A receiving waveform sampled by an ADC is temporarily stored in a memory which is a part of making up the beamformer, and a part of time delay operation is implemented by utilizing the difference between write address and read address of the memory. In the current semiconductor technology, computing operation of a LSI device for implementing the beamformer is operable at an operating frequency of at least 30 MHz to 500 MHz compared with the sampling frequency of an ADC (approximately 15 MHz to 80 MHz). It is possible to realize a circuit which can handle sampled signals of an ADC with multiple channels, as well as simultaneously perform the processing of multiple beams. While these are realized by time divided multiple access, when for example performing, by time division, the reception from directions having a large angular difference, there is no continuous relationship in addressing delays in the memory, and therefore the delay data becomes discontinuous (hopping) by time division. Under such circumstances, it is difficult to realize the time divided multiple access by continuous increase and decrease of the read address to the memory for storing sampled signals of the ADC as disclosed in U.S. Pat. No. 5,522,391. Further, when considering the above described time divided multiple access, it is more efficient, for the beamformer data, to perform a control to update the coefficients of computation function in a common time span (interval) within an intended computation accuracy, but this is not known in conventional art.

As a consequence, the beamformer data for realizing a high accuracy or a high performance is typically in a table look-up format by using storage means such as a memory. The beamformer data requires the preparation of different data of typically 128 to 512 types in various beam directions especially in 2-dimensional sector scanning and trapezoidal scanning. In order to realize a high dynamic range with little distortion, it is necessary to provide a large number of divided sections in the range direction so as to provide smoothly jointed beamformer data.

It is an object of the present invention to store beamformer data, which cannot be represented by analytic functions comprised of few terms, and which is a multivariate nonlinear function dependent on multiple variables which is numerically set, with its total data amount compressed within a predetermined approximation accuracy, and to create time dependent data smoothly in time.

### Means for Solving the Problems

The diagnostic ultrasound imaging system of the present invention comprises an ultrasonic beamformer which provides a different delay time and weighting for response to a plurality of transmit and receive elements of ultrasound transducer to transmit/receive ultrasound into and from a living body, in which the ultrasonic beamformer comprises numerical computation means which sequentially iterates the processing to compute beamformer data made up of the delay times and weighting for response by using a function including a combination of linear terms of plural variables with quadratic term of a time variable within a partitioned time span. The numerical computation means may sequentially iterate the processing to compute delay times and weighting for response by using a function including a combination of linear term of plural variables, quadratic terms which are products of time variable and one of the plural variable, and quadratic term of a time variable within a partitioned time span.

### ADVANTAGES OF THE INVENTION

According to the present invention, it is possible to reduce the distortion of output signal due to the discontinuities of phase and amplitude at junction points of span in the time direction of the beamformer data and to reduce the total amount of data.

### BRIEF DESCRIPTION OF THE DRAWINGS

FIG. 1 is an explanatory drawing to show the configuration of a beamformer data computing unit.

FIG. 2 is an explanatory drawing of the dependency on time-division frame processing of computation.

FIG. 3 is an explanatory drawing of a time division frame for computation.

FIG. 4 is an explanatory drawing of the data format of the beamformer data computing unit.

FIG. 5 is an explanatory drawing of an application example to 3-dimensional beamformer computing.

FIG. 6 is an explanatory drawing of a solving solution of delay data by a linear programming.

FIG. 7 is an explanatory drawing of a solving solution of weighing data by a linear programming.

FIG. 8 is an explanatory drawing of a linear programming solving solution of delay data including a continuity condition.

FIG. 9 is an explanatory drawing of a linear programming solving solution of weighing data including a continuity condition.

FIG. 10 is an explanatory drawing of frequency-band division beamforming computation.

FIG. 11 is an explanatory drawing of the configuration of a medical ultrasound imaging system equipped with the beamformer data computing unit of the present invention.

FIG. 12 is an explanatory drawing of delay data.

FIG. 13 is an explanatory drawing of weighing data.

### DESCRIPTION OF SYMBOLS

400 Probe
410 Transmit and receive transducer elements
100 Beamformer data computing unit
200 Transmitting circuit
300 Transmit/receive switch circuit
210 Receiving circuit
120 Receiving beamformer
121 Beam output signal
130 Scan converter
131 Video signal
140 Display means
TCPU Processor
EXTRAM Storage means
TW Transmit sound wave
RW Receive sound wave
τ Delay data
w Weighing data
S1 to Sk Time span
spn1 to spnk Span size
RSPN Span size register
TA Accumulated time after calculation of “beamformer data” starts
Tₛ Time counting value in a time span
SQGN Time divided multiple access sequencer
1spn Index number of spans
SQLD Coefficient sequencer
CH Number of channels
BM Number of beams
BMmax Maximum value of the number of beams BM
M Maximum length of time divided multiple access frame
RP1, RP2, . . . , RPn Parameter variable register
p11 to p1m, p21 to p2m, pn1 to pnm Parameter values
PA Bus of processor
RAP Aperture masking-time register
ap1 to apm Stored values of aperture masking-time register
V_{τ} Delay data function
V_{w} Weighing data function
P₁ to Pₙ Parameter variables
A_{τ1}, A_{w1} 2nd-degree coefficient of intra-span time Tₛ
A_{τ2}, A_{w2} 1st-degree coefficient of intra-span time Tₛ
B_{τ1} to B_{τn}, B_{w1} to B_{wn} Coefficients of 2nd-degree products of intra-span time Tₛ and parameter variables P₁ to Pₙ
C_{τ1} to C_{τn}, C_{w1} to C_{wn} 1st-degree coefficients of parameter variables P₁ to Pₙ
D_{τ}, D_{w} Initial constant terms
RADR Beam channel designation register
adr1 to adrm Index values stored in beam channel designation register
SQLD Coefficient sequencer
CD Read-out data output from storage means EXTRM
RACM Accumulating register
RA1, RA2, RB1 to RBn, RC1 to RCn Coefficient registers
M Time divided multiple access frame length
a11 to a1m Stored values of coefficient register RA1
a21 to a2m Stored values of coefficient register RA2
b11 to b1m, b21 to b2m, . . . , bn1 to bnm Stored values of coefficient registers RB1 to RBn of B₁ to Bₙ
c11 to c1m, c21 to c2m, . . . , cn1 to cnm Stored values of coefficient registers RC1 to RCn of C₁ to Cₙ
SQGN Time divided multiple access sequencer
MPYTT, MPYA1, MPYA2, MPYP1 to MPYPn, MPYC1 to MPYCn Multiplication means
A1TT A₁·Tₛ² computing term
A2T A₂·Tₛ computing term
BP1, BP2, . . . , BPn Outputs of B1·P1·Tₛ, B2·P2·Tₛ, . . . , Bn·Pn·Tₛ computing terms
CP1, CP2, . . . , CPn Outputs of C₁·P₁, C₂·P₂·Tₛ, . . . , Cn·Pn computing terms
SUM Summer
ACUM Accumulator
SEL1, SEL2 Selector
LD Instructed output of Time divided multiple access sequencer SQGN
CMP Comparator
DG Masking command output of comparator
DAT Beamformer data computation output
RA Reference address bus of SQGN
Tx Transmit synchronize reference signal
TxP Transmit interval
LDS Time span load signal
FR1 Last time-division frame period in time span S1
NCH Channel number
NBM Beam number
FMT1, FMT2 Beamformer data format
ψ, φ Beam steering direction angle
s(sx, sy, sz) Reference point coordinates of beam sound axis
NA Reference axis
O(x, y, z) Coordinate system
NB Acoustical beamforming direction
N_{LU}, N_{UU}, N_{UL}, N_{LL} Angular span edge of interpolating calculation span of beam data
NB Acoustical beamforming direction
R₁, R₂, R₃ Axial range along acoustical beam
(x′, y′, z′) Coordinate system with reference axis NA as z′-axis
f₁ to f₅ Functions
MDT(TA) Ideal delay data function
MDW(TA) Ideal weighing data function
ε_{τ} Allowable error for MDT(TA)
ε_{w} Allowable error for MDW(TA)
ETU(TA) Allowable upper limit function of delay time τ
ETL(TA) Allowable lower limit function of delay time τ
EWU(TA) Allowable upper limit function of weighting w
EWL(TA) Allowable lower limit function of weighting w
511 to 51k, 521 to 52k Optimal function curves within a span
ε_{Jτ}, ε_{Jw} Allowable error at a time of a junction point of span
U_{Eτh}, U_{Ewh} Values of first partial derivative with respect to time at a time of a junction point of span
ε_{Dτh}, τ_{Dw} Allowable difference of values of first partial derivative with respect to time at a time of a junction point of span
BND Full bandwidth of signal through transmit and receive
BD0 to BD3 Partial band
DP1, DP2 Depth range in the body
ATT1, ATT2 Attenuation

### BEST MODE FOR CARRYING OUT THE INVENTION

The present invention is provided with sum-of-product computation means which utilizes a function having a linear combination term of a quadratic function of time in which time is divided into multiple segments and linear functions of any variable other than time, for the computation of transmitting or receiving beamformer data.

The above described computation means can achieve an efficient data generation by performing the computation in which the control of generation means of time term is made common so that time segmentation of beamformer data is made common. Further, adding a constant adding term in the time span which is the first in the processing order in the above described computation, and in the subsequent spans, performing computation for accumulating onto the last value of the previous span will enable to omit the updating of the constant term for each time span.

Further, by retaining coefficients or initial values, which are included in the functions of each span, in storage means in the computation of time segment, and reading out them from the storage means prior to the start of computation of each span, it is made possible to optimize the transfer amount from the storage means to the computation means for the same configuration.

Further, it is also possible to select a read-out operation which utilizes data multiplexing means to the computation means to omit read-out operation of coefficients other than the initial values and coefficients of time-wise linear function and thereby to realize beamformer data with a minimum configuration.

Further, by making the above described time span common for every predetermined number of channel processing and every predetermined beamforming condition of the beamformer, the need of separately acquiring and controlling individual time-span size information is obviated thus enabling the means to be simplified.

Further, by making the above described time span common for every predetermined number of channel processing and every predetermined beamforming condition of the beamformer, the need of separately acquiring and controlling individual time-span size information is obviated thus enabling the control means for fitting a function to be simplified.

Further since the change amount (increment, etc.) to be added to the computation of time function of each span is changed for every predetermined number of channel processing and every predetermined beamforming condition in the time segment, it is possible to perform computation using the same time variable value in the final computation of span and to obviate the need of preparing multiple beamformer data, achieving efficiency gain.

Further since the initial value of a time variable to be added to the computation of time function of each span is changed for every predetermined number of channel processing and every predetermined beamforming condition in the time segment, it is possible to perform computation using the same time variable value in the final computation of span and to obviate the need of preparing multiple beamformer data, thereby achieving efficiency gain.

Further, since time segments are segmented at an unequal interval for use and, in accordance with a time position at which beamformer data rapidly changes and a time position at which beamformer data slowly changes, a short time span is adopted for the former position and a longer time span is adopted for the latter position, it is possible to reduce the total number of time spans thereby reducing the beamformer data amount.

Further, by arranging non-time, multiple linear function terms of computation to be in accordance with the function of variables which specify the transmitting/receiving direction of beamformer data and treating it as a partial differential coefficient of the change of the beamformer data, it is possible to efficiently treat the beamformer data.

Further, it is possible to determine the function computation coefficient terms for every time span by a linear programming within a desired error range, and to conveniently define accuracy conditions when various independent parameters are incorporated.

Further, by arranging that time-wise 1st differential coefficients to substantially correspond to each other at a junction point between time spans, it is possible to reduce the output fluctuation of the beamformer output at a connecting portion of time spans to a level at which there will be no problem in practical use.

Further, by assigning time divided multiple access to divided signal bands and providing computation coefficients of beamformer independently for every signal band, it is possible to obtain frequency dependent beamformer output.

Further, by performing disabling processing of beamformer data output by the comparison between a time counter other than that of time span and time length, it is possible to set a time at which input channel data starts being involved in the beamformer independently from the beamformer data settings optimized for the time span.

Further, providing means for providing a fixed time value at an intra-span time corresponding to the focal point position at one time in transmission, it is possible to make beamformer data for transmission and reception to be shared.

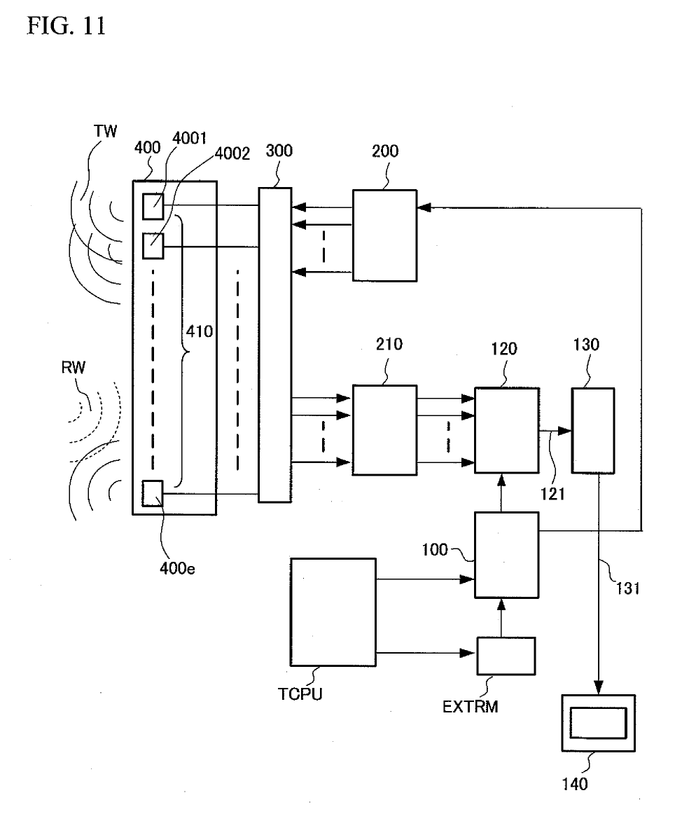

FIG. 11 shows a configuration example of the diagnostic ultrasound imaging system to which the present invention is applied. A probe 400 for transmitting/receiving ultrasound includes built-in transmit and receive transducer elements 410 made up of a number e of transmit and receive transducer elements 4001, 4002 to 400e for performing electroacoustic conversion. The transmit and receive transducer elements 4001, 4002 to 400e are often made up of a piezoelectric substance such as a ferroelectric material which is caused to generate pressure by applied voltage upon transmitting ultrasound, and to generate voltage by ultrasonic pressure upon receiving it. As is widely known, imaging in a diagnostic ultrasound imaging system is performed such that a transmit sound wave TW is radiated into a living body which is the object to be inspected by applying different voltage waveforms to the transmit and receive transducer elements 410, and receive sound wave (echo) RW reflected from desired points in the living body are compensated for the propagation time difference and phase difference and summed up to reconstruct an image in the living body. The reflected signals from desired points are correctly compensated for the phase with one another and are summed up intensifying one another, and the reflected signals from undesired directions interfere with one another and are suppressed by being summed up so that an acoustic beam is formed and thereby information in the living body is scanned and visualized. The means for compensating for the propagation time difference in transmission or reception is called as a beamformer (phased array).

The transmission of ultrasound is performed by causing a transmitting circuit 200 to supply a transmission voltage having a pulse, a burst, and a frequency-sweeping waveform to the transmit and receive transducer elements 410 within the probe 400 via a transmit/receive switch circuit 300, in accordance with the output information of the transmission time and amplitude intensity outputted by a beamformer data computing unit 100. The transmit and receive transducer elements 410 convert the voltage into pressure so that ultrasound is transmitted. The ultrasound transmitted from the probe 400 is reflected at the interior of the object to be inspected to become a reflected wave, and is again received by the transmit and receive transducer elements 410 of the probe 400 so that pressure is converted into voltage and becomes an input to a receiving circuit 210 via a transmit/receive switch circuit 300. In the receiving circuit 210, amplification and band limiting are performed and signals of multiple channels are supplied to a receiving beamformer 120. At the receiving beamformer 120, a beam output signal 121 in which time and amplitude are combined to realize a directivity according to the output information of the delay time and amplitude weighting outputted by a beamformer data computing unit 100. The beam output signal 121 becomes an input to a scan converter 130, and is converted into a video signal 131 so that a real-time cross-sectional image and a stereoscopic model are displayed on display means 140. The processor TCPU not only controls the entire equipment, but also performs the writing of beamformer data for associated storage means EXTRAM via the beamformer data computing unit 100. The storage means EXTRM can be implemented for example by using a Static Random Access Memory (hereafter, referred to as SRAM).

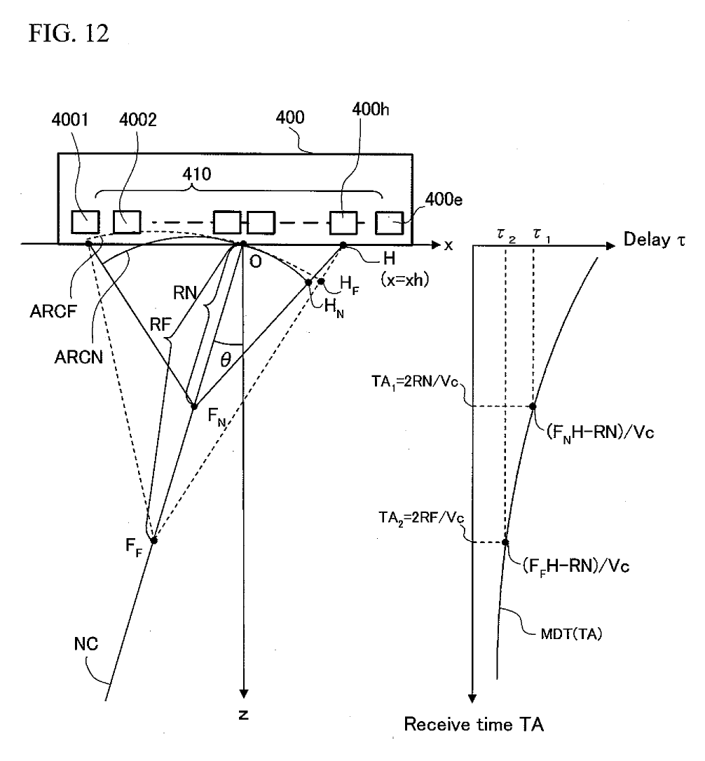

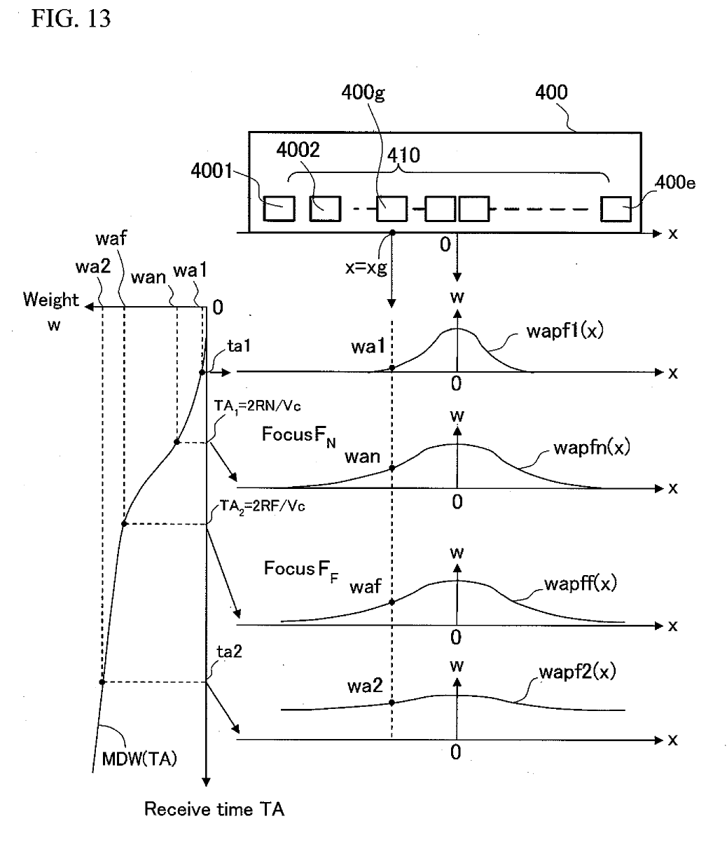

Next, an example of the beamformer data will be described taking an example in which the probe 400 is of a sector scanning type, with reference to FIGS. 12 and 13.

An example of the actual method of determining delay data τ will be described with reference to FIG. 12. A coordinate system is determined such that the contact surface (for example a body surface of human) between the probe 400 and the object to be inspected includes the x-axis of orthogonal coordinates O-xyz and the depth direction inside the object to be inspected coincides with the z-axis. The y-axis is assumed to be the direction normal to the plane of the figure. In general, the x-axis indicates an azimuth (sector angle) direction and the z-axis indicates a range (depth) direction. The coordinate origin O is set to be at the center of the transmit and receive transducer elements 410 of the probe 400. In sector scanning, a raster NC for performing the transmission/reception of ultrasound and acquiring reflected wave information is assumed to be radial, and is iterated while successively changing the azimuth angle θ, which is the steering direction angle formed with the z-axis, to form a fan-shaped scanning image. Upon transmission of ultrasound, a pulse is transmitted in advance so as to form a focal point or focal zone including points on NC. Upon reception of reflected signals, it is assumed that the receive focal point dynamically moves on NC successively from the vicinity of the x-axis to the maximum depth to be scanned. The operation in which the focal point or focal zone upon reception is successively modified while changing in time is widely known as the dynamic receive focusing.

Now consider receive focal points F_{N}, F_{F} which are determined by the ranges RN, RF on NC with reference to the coordinate origin O. Then, consider to perform the calculation of the time varying delay data τ, which should be given to specific elements 400h out of the transmit and receive transducer elements 4001, 4002 to 400e, with reference to point H on the x-axis. In a time series of received signals of the elements 400h, waveform data at a time point, which is relatively different by the amount of delay data τ, will be computed by a beamformer. Letting H_{N} be the point at which an arc ARCN having a radius of RN, which is set with respect to the receive focal point F_{N}, intersects with the line F_{N}H, since the length of the line section F_{N}H_{N} is equal to RN, delay data τ₁, which is the time delay amount to be given to the received signals of the elements 400h, is determined from a propagating path difference F_{N}H−RN and sound velocity V_{C} of the object to be inspected, as τ₁=(F_{N}H−RN)/V_{C}. In the dynamic receive focusing which changes with time, it is possible to determine “a receiving time TA” with virtual transmit time of a transmit wave at the coordinate origin O being 0.

It is noted that the acquisition of the received signals of all the transmit and receive transducer elements 4001, 4002 to 400e are supposed to start at appropriate time with the condition of TA<0. The receiving time TA at which a delay data τ₁ should be given to the elements 400h is supposed to be TA₁=2RN/V_{C} with the reference to the round-trip time of the soundwave on NC from the start of reception. Similarly, letting H_{F} be the point at which an arc having a radius of RF, which is set with respect to the receive focal point F_{F}, intersects with the line segment F_{N}H, the delay data τ₂ at that time will be τ₂=(F_{F}H−RF)/V_{C}. The receiving time TA at which the delay data τ₂ should be given to the elements 400h will be TA₂=2RN/V_{C} with reference to the round-trip time of the soundwave on NC. Since the delay data τ is continuously given in the dynamic receive focusing, an ideal delay data function MDT(TA), which is a function curve of the delay data τ with respect to the receiving time TA, is calculated. The ideal delay data function MDT(TA) is different for each of the transmit and receive transducer elements 4001, 4002 to 400e and further, in the case of sector scanning, it is usual that the ideal delay data function MDT(TA) differs every time the azimuth angle θ of the raster NC is changed in scanning.

Next, an example of actual setting of weighing data w (apodization coefficient data) which is set along with the delay data τ will be shown in FIG. 13. The x-axis is supposed to be similar to the case of FIG. 12. For each transmit focal point or each focal point position of the dynamic receive focusing, functions of weighing data w depending on the element position x to be given to each of the transmit and receive transducer elements 4001, 4002 to 400e are predetermined. These functions are usually calculated in advance so as to satisfy desired characteristics in accordance with the purpose considering an effective aperture width with respect to the directivity, sensitivity, and focal distance of the beam to be formed. In general, in a typical B mode imaging, in a case in which the focal distance is present very close to the surface of the probe, weighting of which absolute value is nonzero is assigned to part of elements in the vicinity of the center of the aperture, and in many cases, weighting of 0 is assigned to the elements near the both ends of the aperture. Further, as the focal distance increases, the range of the elements to which non-zero weighting is assigned is widened. Since when the effective aperture size is too large with respect to the focal distance, the degree of inclusion of undesired transmission/reception responses from other than the vicinity of the intended focal point increases, it is practice to limit an effective aperture size. This limitation can be implemented by making the weighing data w be 0, and by separately preparing time information to give significant weighing data w first in time to set a span in which the delay data τ and the weighing data w are disabled.

It is general that the weighting to be given to each of the elements is adapted to be a smooth and continuous function with respect to receiving time TA. This is because a large discontinuity may lead to a discontinuity in time in the gain and directivity of the beamformer output unless special consideration is given adjusting the weighting of all the elements. FIG. 13 illustrates weighting functions in the aperture direction: wapf1(x), wapfn(x), wapff(x), wapf2(x) which depend on the aperture position x at specific times ta₁, TA₁, ta₂, TA₂ of receiving time TA. As such weighting functions in the aperture direction, Gauss window, Hamming window, Hanning window, a rectangular (boxcar) window, or other window functions are set for intended purpose. In the example in FIG. 13, weighing data w to be given to the transmit and receive transducer elements 400g which are located at the position of x=xg at times tab, TA₁, ta₂, and TA₂ are wa1=wapf1(xg), wan=wapfn(xg), waf=wapff(xg), and wa2=wapf2(xg). Since, in the dynamic receive focusing, the weighing data w is given continuously in time, an ideal weighing data function MDW(TA) of the weighing data w with respect to the receiving time TA is calculated. It is general that the ideal weighing data function MDW(TA), as with the ideal delay data function MDT(TA), differs for each of the transmit and receive transducer elements 4001, 4002 to 400e, and also differs every time the azimuth angle θ of the raster NC is changed by scanning.

As so far described, the delay data τ and the weighing data w are generated for every channel for transmitting/receiving ultrasound, and for every beam to be formed corresponding to the position of the raster NC.

In the above described computation function, by arranging the computation function of the beamformer data for transmission or reception to be function which is a linear combination of: 2nd- and 1st-degree time term which is segmented in time; 2nd-degree term made up of the product between time and variable other than time; 1st-degree term and a constant term made up of time and variable other than time, it is possible to store the beamformer data which is a non-linear function depending on multiple variables with a linear sum of polynomial function comprised of few terms within a predetermined approximation accuracy while efficiently compressing the total data amount, and to generate time dependent data smoothly in time. With the head of time segment being 0, time counted by the unit of computation clock period is intra-span time T_{S}.

Let v_{τ} be an approximate function of the delay data τ and v_{w} be the approximate function of the weigh data w. These approximate functions are generated by the computation function shown below, with intra-span time T_{S} within a time span and parameter variables P₁ to Pₙ being variables, using 2nd-degree coefficients A_{τ1}, A_{w1} of intra-span time T_{S}, 1st-degree coefficients A_{τ2}, A_{w2} of intra-span time T_{S}, coefficients B_{τ1} to B_{τn} and B_{w1} to B_{wn} of 2nd-degree products between intra-span time T_{S} and parameter variables P₁ to Pₙ, 1st-degree coefficients C_{τ1} to C_{τn} and C_{w1} to C_{wn} of parameter variables P₁ to Pₙ, and initial constant terms D_{τ}, D_{w}.

$${v}_{τ} ( Ts , {P}_{1} , {P}_{2} , … , {P}_{n} ) = {A}_{τ 1} {T}_{S}^{2} + {A}_{τ 2} {T}_{S} + {\sum }_{i = 1}^{n} ( {B}_{τ i} \cdot {T}_{S} \cdot {P}_{i} + {C}_{τ i} \cdot {P}_{i} ) + {D}_{τ} \\ {v}_{w} ( Ts , {P}_{1} , {P}_{2} , … , {P}_{n} ) = {A}_{w 1} {T}_{S}^{2} + {A}_{w 2} {T}_{S} + {\sum }_{i = 1}^{n} ( {B}_{wi} + {T}_{S} \cdot {P}_{i} + {C}_{wi} \cdot {P}_{i} ) + {D}_{w} \qquad [ Expression 2 ]$$

The coefficients A_{τ1}, A_{w1}, A_{τ2}, A_{w2}, B_{τ1} to B_{τn}, B_{w1} to B_{wn}, C_{τ1} to C_{τn}, C_{w1} to C_{wn}, and the initial constant terms D_{τ}, D_{w} are stored in storage means EXTRM for every k time spans, for each channel number CH, and for each beam number BM. It is noted that the initial constant terms D_{τ}, D_{w} may be limited only in the first time span.

The superiority of the arrangement that the approximate function is quadratic (second degree) at least with respect to time will be shown by way of calculation examples with the approximate function v_{τ} of the delay data τ being an example.

Let the intra-span time T_{S} within the time span divided by the above described approximation equation be the elapsed time from the start point of two forward and backward time spans which end or start at a specific time TA_{J} of the receiving time TA in FIG. 12. Let all the coefficients excepting the time-wise 1st-degree coefficient A_{τ2} and the initial constant term D_{τ} be 0 (A_{τ1}=0, B_{τ1} to B_{τn}=0, and C_{τ1} to C_{τn}=0), and a piecewise linear function which depends on time alone will be hereafter referred to as a piecewise linear function computing equation PWLE(Tₛ).

PWLE(Ts)=A_{τ2}T_{S}+D_{τ}

This is called a Piece-Wise Linear (PWL) function since it is divided into multiple spans and is segmented by 1st-degree linear functions, and is widely known in general. The approximation by a PWL function is the simplest, and that is considered to be an approximate function in which the number of computing coefficients needed for one span is minimum. Further, a piecewise quadratic function which depends only on time and in which other than 2nd-degree coefficient A_{τ1} of intra-span time Tₛ, 1st-degree coefficient A_{τ2} and the initial constant term D_{τ} are 0 (B_{τ1} to B_{τn}=0, and C_{τ1} to C_{τn}=0) is hereafter referred to as a piecewise quadratic function computing equation PWQE(Tₛ).

PWLE(Ts)=A_{τ1}T_{S}²+A_{τ2}T_{S}+D_{τ}A^{τ1},

where A_{τ2} and D_{τ} are successively set with different values for each time span.

As a concrete example of calculation, let the frequency of transmit wave be 5 MHz, sound velocity Vc=1540 m/s, azimuth angle θ=0 radian, and the x coordinate value of the point H representative of the position of the elements 400h be xh=2.464 mm. Study was made on a case in which at time TA_{J} at which receiving time TA becomes 2.12 μs on the raster NC, time spans of successive two piecewise linear function computing equations or a piecewise quadratic function computing equation are connected. By a numerical calculation with a sufficient numerical accuracy, first, an ideal delay data function MDT(TA) with respect to TA of delay data τ is calculated. The ideal delay data function MDT(TA) is, as described in FIG. 12, calculated from the propagating path length difference of sound wave and the sound velocity Vc, where the length difference can be geometrically calculated from the position of receive focal point (F_{N} at TA=TA₁ and F_{F} at TA=TA₂, etc.) on the NC, which is dynamically assumed time-wisely, and element position xh representative of the point H. An allowable error when performing the approximation to the obtained ideal delay data function MDT(TA), delay time accuracy ε_{τ} is set to be 1/512 of the period of 5 MHz.

With ε_{τ}>0, an error of ±ε_{τ} is allowed for the ideal delay data function MDT(TA). As a result of this, an allowable upper limit function ETU(TA) and an allowable lower limit function ETL(TA) of the delay data τ are defined.

ETU(TA)=MDT(TA)+ε_{τ}

ETL(TA)=MDT(TA)−ε_{τ}

Coefficients are searched so that approximate values of a piecewise linear-function computing equation and a piecewise quadratic-function computing equation for two spans, of which a junction point is at TA_{J}=2.12 μs, are included between ETL(TA) and ETU(TA).

To be more specific, PWLE₁(T₁₁) defined by time variable T₁₁=TA−TA_{S1} is set with respect to the time span TA_{S1}≦T₁₁≦TA_{J}; PWLE₂(T₁₂) defined by time variable T₁₂=TA−TA_{J} with respect to the time span TA_{J}≦T₁₂≦TA_{E1}; PWQE₁(T₂₁) defined by time variable T₂₁=TA−TA_{S2} with respect to TA_{S2}≦T₂₁≦TA_{J}; and PWQE₂(T₂₂) defined by time variable T₂₂=TA−TA_{J} with respect to TA_{J}≦T₂₂≦TA_{E2}, and under the following restrictive condition:

ETL(TA)≦PWLE₁(T₁₁), PWLE₂(T₁₁)≦ETU(TA)

ETL(TA)≦PWQE₁(T₂₁), PWQE₂(T₂₂)≦ETU(TA)

a search has been made for coefficients such that TA_{S1} and TA_{S2} are the minimum time span, and TA_{E1} and TA_{E2} are the maximum time span. A numerical computation search has been conducted supposing that approximate functions PWLE₁(T₁₁) and PWLE₂(T₁₁) or PWQE₁(T₂₁) and PWQE₂(T₂₂) of two successive spans have a junction point ETU(TA_{J}).

As PWLE₁(T₁₁), a span of a size of 0.468 μs, in which TA_{S1}=1.652 μs, has been obtained, and as PWLE₂(T₂₁), a span of a size of 0.492 μs, in which TA_{E1}=2.612 μs, has been obtained. As PWQE₁(T₂₁), a span of a size of 2.075 μs, in which TA_{S2}=0.045 μs, has been obtained, and as PWQE₂(T₂₂), a span of a size of 2.846 μs, in which TA_{E2}=4.966, has been obtained. The sum of the sizes of two successive spans is 0.96 μs in the piecewise linear-function computing equation, and 4.92 μs in the piecewise quadratic-function computing equation, showing that the piecewise quadratic-function computing equation can approximate a time span size four times as much as that of the piecewise linear-function computing equation. As a result of this, it is possible in some conditions to reduce the number of time spans to not more than half by the piecewise quadratic-function computing equation. It is noted that in reality, intra-span time T_{S} which is obtained by normalizing T₁₁, T₁₂, T₂₁, and T₂₁ by a beamformer data computing period, and coefficients of the piecewise quadratic-function computing equation and the piecewise linear-function computing equation normalized by a required accuracy of the delay time are determined at an accuracy needed for internal computing.

D_{τ} which is used in the piecewise quadratic-function computing equation and the piecewise linear-function computing equation can be computed by successively accumulating the computed value of the final value of the adjoining, immediately preceding time span so as to be the initial-value constant term D_{τ} of the following time span. In the internal computing accuracy, as a specific example, the bit accuracy of significand for the coefficients A_{τ1}, A_{τ2}, D_{τ} will be sufficient in reality if it is 23 bits. Since if the same data amount of IEEE 32 bit single precision binary floating-point format is assigned to each coefficient, for each increase of time span, the piecewise quadratic-function computing equation needs 32+32=64 bits for A_{τ1}, A_{τ2} and the piecewise linear-function computing equation requires 32 bits for A_{τ2}, if the number of spans is appropriately large, generally, there is no need of considering the data amount of D_{τ} which will be used only in the first time-span computing, and although the data amount of each piecewise quadratic-function computing equation will be doubled compared with the data amount of the piecewise linear-function computing equation, the number of time spans can be more than halved enabling to decrease the total data amount which is the product thereof.

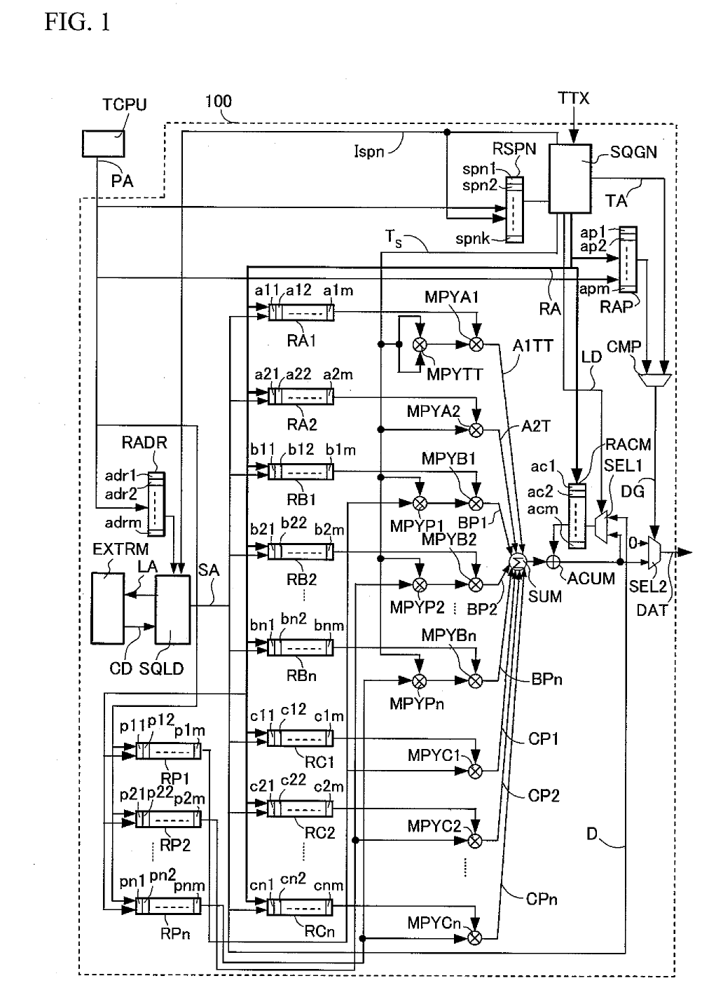

In a function which has a monotonous change with respect to the increase of TA such as the ideal delay data function MDT(TA) of FIG. 12, focusing on a certain junction point, it is obvious also from the above described example that a piecewise quadratic-function computing equation can approximate a wider time range of TA than a piecewise linear-function computing equation within a desired delay time accuracy ε_{τ}. Even if the function is not a monotonous one, but one having poles and inflection points, by selecting such a peculiar point as the junction point of span and 2nd degree, using a piecewise quadratic-function computing equation is more advantageous than using a conventionally known piecewise linear-function computing equation because the necessary storage volume of the storage means EXTRM of FIG. 1 can be reduced.

An effective piecewise approximation for the ideal delay data function MDT(TA) relating to the delay data τ of FIG. 12 can be similarly implemented regarding the piecewise approximation of the ideal weighing data function MDW(TA) relating to the weighing data w of FIG. 13.

Further, as an example of more sophisticated embodiment, even when an approximate equation is arranged to use coefficients B_{τ1} to B_{τn} and C_{τ1} to C_{τn} reflecting the dependence on the parameter variables P₁ to Pₙ other than time, by admitting a predetermined allowance of approximation error, it is similarly true that the coefficient data amount can be effectively reduced. Description will be made on a case in which as the first parameter P₁, P₁ is arranged to correspond to an azimuth angle θ (beam steering direction angle) and the second parameter P₂ is arranged to correspond to the sound velocity Vc. First, the angle range of the azimuth angle θ is divided into multiple spans. As an index corresponding to an arbitrary angle θᵢ (θa≦θi≦θb) between a start angle θa and an end angle θb of one divided span, a parameter variable P₁=(θi−θa)/(θb−θa) is defined. Further, as an index corresponding to an arbitrary sound velocity (Vca≦Vci≦Vcb) between a specific set range [Vca, Vcb] of sound velocity Vc, a parameter variable P₂=(Vci−Vca)/(Vcb−Vca) is defined.

The ideal delay data function MDT(θ, Vc, TA) and the ideal weighing data function MDW(θ, Vc, TA) which have no error relating to the azimuth angle θ, sound velocity Vc, and receiving time TA are calculated within spans [θa, θb] and [Vca, Vcb]. Using a delay time accuracy ε_{τ} the allowable upper limit function ETU(θ, Vc, TA)=MDT(θ, Vc, TA)+ε_{τ} and the allowable lower limit function ETL(τ, Vc, TA)=MDT(τ, Vc, TA)−ε_{τ} of the delay data are calculated. Using a weighting accuracy ε_{τ}, the allowable upper limit function EWU(θ, Vc, TA)=MDW(θ, Vc, TA)+ε_{w} and the allowable lower limit function EWL(τ, Vc, TA)=MDW(θ, Vc, TA)−W of the weighing data are calculated. It is possible to determine B_{τ1}, C_{τ1}, B_{τ2}, C_{τ2}, B_{w1}, C_{w1}, B_{w2}, and C_{w2} such that the following approximate function, which use B_{τ1}, C_{τ1}, B_{τ2}, C_{τ2}, B_{w1}, C_{w1}, B_{w2}, and C_{w2}, VTAU(P₁, P₂, Tₛ) and VWEI(P₁, P₂, Tₛ):

VTAU(P₁,P₂,Tₛ)=A_{τ1}T_{S}²+A_{τ2}T_{S}+B_{τ1}P₁T_{S}+B_{τ2}P₂T_{S}+C_{τ1}P₁+C_{τ2}P₂+D_{τ}

VWEI(P₁,P₂,Tₛ)=A_{w1}T_{S}²+A_{w2}T_{S}+B_{w1}P₁T_{S}+B_{w2}P₂T_{S}+C_{w1}P₁+C_{w2}P₂+D_{w}

satisfy ETL(τ, Vc, TA)≦VTAU(P₁, P₂, Tₛ)≦ETU(θ, Vc, TA) and EWL(τ, Vc, TA)≦VWEI(P₁, P₂, Tₛ)≦EWU(θ, Vc, TA).

As a result of this, while it becomes possible to provide focus data through interpolating calculation by parameter variables P₁, P₂ despite that it has been necessary to independently prepare delay data and weighing data every time the azimuth angle θ and the assumed sound velocity Vc are changed, there is an obvious advantage that the necessary storage volume of the storage means EXTRM can be dramatically reduced. By adding a function according to computation terms B_{τ1}P₁Tₛ, B_{τ2}P₂Tₛ, B_{w1}P₁Tₛ, B_{τ2}P₂Tₛ relating to both the parameter and time in addition to the approximate function according to only computation terms of C_{τ1}P₁, C_{τ2}P₂, C_{w1}P₁, C_{w2}P₂ only according to parameter variables P₁ and P₂, it is made possible to further enlarge the size of the spans [θa, θb] and [Vca, Vcb] for performing approximation when compared for the same error allowance, consequently reducing the necessary storage volume of the storage means EXTRM. Thus, it is possible to store such numerically set beamformer data which is a multivariate non-linear function dependent on multiple variables, by compressing the data amount within a predetermined approximation accuracy and to generate time dependent data as a smooth function. Although it is easily inferred from analogy that as a piecewise approximation by a common multivariate non-linear function, a maximum of 2nd total differentiation can be used as a multivariate Taylor expansion, when considering the computation order of the delay data τ and the weighing data w of usual beamformer data within the beamformer computation, it is characteristic that in view of the fact that the imaging in a medical ultrasound imaging system is achieved by iterating multiple transmission/reception, by predetermining piecewise span of variables other than time, simultaneously setting a common allowable error in a piecewise span of variables other than all the time variables, and finally expanding the segment length of time span, the total amount of beamformer data is reduced by independently determining a desired approximate function in a time segment, thereby compressing the necessary storage volume of the storage means EXTRM.

Further, D_{τ} and D_{w} for each span can be approximately computed by performing successive accumulation such that the final computed value of adjacent, immediately preceding span becomes the initial value constant terms D_{τ} and D_{w} of the following span. As a result of this, even when parameter variables are included, there is no need of using D_{τ} and D_{w} in computation for each time span of TA excepting the initial time span, and it is possible to reduce the necessary storage volume of the storage means EXTRM. Without being limited to parameter variables P₁ and P₂ corresponding to the above described azimuth angle θ and the assumed sound velocity Vc, and including any other parameter terms, it is obvious that the necessary storage volume of the storage means ESTRM can be reduced. Further by using the time span segmented into unequally spaced intervals, it is possible to effectively reduce the total number of divisions of the time span, and to perform the computation suitable for the dynamic receive focusing by configuring that the division is commonly performed for parameter variables P₁ to Pₙ other than time. Further, in the case of maintaining a predetermined accuracy by function computation, when beamformer data rapidly changes with time, it is possible to effectively set a short time span only in the portion concerned.

Next, an embodiment of the beamformer data computing unit 100 of the present invention will be described with reference to FIG. 1. The coefficients τ₁, A_{w1}, A_{τ2}, A_{w2}, B_{τ1} to B_{τn}, B_{w1} to B_{wn}, C_{τ1} to C_{τn}, C_{w1} to C_{wn} of the approximate function v_{τ} of the delay data τ, the approximate function V_{w} of the weighing data w and the initial constant terms D_{τ} and D_{w} are stored in the storage means EXTRM for every k number of time spans, every channel number CH, and every beam number BM.

v_{τ} and v_{w} are functions which change with intra-span time Tₛ within a time span which is obtained by dividing the receiving time TA, and the receiving time TA is divided into a total of k sections, that is, time spans S1 to Sk. The sizes of time span S1 to Sk are counted by the unit of computing clock period and are caused to be stored in a span size register RSPN by a processor TCPU respectively as k span sizes spn1 to spnk data. With the receiving time TA from the start of beamformer data computation, and the lead of each time span being 0, the intra-span time Tₛ which is counted by the unit of computing clock period is calculated and outputted by a time divided multiple access sequencer (controller) SQGN.

The time divided multiple access sequencer SQGN outputs an index number of spans Ispn, and successively refers the span sizes from spn1 to spnk from the span size register RSPN. The index number of spans Ispn is also supplied to a coefficient sequencer (controller) SQLD.

The processing of the beamformer data computing unit 100 is arranged such that a time divided multiple access frame, in which a channel time divided multiple access frame where a computing clock period continues by the number of channels CH, is further iterated by the number of beams BM, is the generation unit of the beamformer data for one time point. Letting the maximum value of beam number BM be BMmax, this will be the maximum length m of the time divided multiple access frame. As a result of this, m=CH×BMmax. The span sizes spn1 to spnk are set to be integral multiples of the maximum length m of the time divided multiple access frame.

In parameter variable registers RP1, RP2, . . . , and RPn, parameter values p11 to p1m, p21 to p2m, and pn1 to pnm corresponding to the maximum length m of the time divided multiple access frame are caused to be stored by the processor TCPU via a bus PA. Similarly, in an aperture masking-time register RAP, stored values ap1 to apm of time lengths to disable the beamformer data computation result are stored via the bus PA.

A beam-channel designation register RADR stores index values adr1 to adrm which refer the channel and beam corresponding to the maximum length m of the time divided multiple access frame. The coefficient sequencer SQLD uses the index values adr1 to adrm and the index number of spans Ispn to read out the coefficients A_{τ1}, A_{w1}, A_{τ2}, A_{w2}, B_{τ1} to B_{τn}, B_{w1} to B_{wn}, C_{τ1} to C_{τn}, C_{w1} to C_{wn} and the initial constant terms D_{τ}, D_{w} from the storage means EXTRM, and calculates and outputs a reference address LA to the storage means EXTRM. The read-out data output CD from the storage means EXTRM converts the data storage format at the storage means EXTRM into a parallel output, and designates storage addresses for an accumulating register RACM and coefficient registers RA1, RA2, RB1 to RBn, and RC1 to RCn, and also reference addresses for beamformer data computation. The accumulating register RACM and the coefficient registers RA1, RA2, RB1 to RBn, and RC1 to RCn can be implemented by using a two-port memory and a register file.

The coefficient sequencer SQLD transfers m number of initial constant terms D for the accumulating register RACM of a storage length m before the start of the first time span S1, and transfers coefficients A_{τ1}, A_{w1}, A_{τ2}, A_{w2}, B_{τ1} to B_{τn}, B_{w1} to B_{wn}, C_{τ1} to C_{τn}, C_{w1} to C_{wn} referenced in time span S1 to the coefficient registers RA1, RA2, RB1 to RBn, and RC1 to RCn of a storage length m. In the last time-division frame period, the coefficients A_{τ1}, A_{w1}, A_{τ2}, A_{w2}, B_{τ1} to B_{τn}, B_{w1} to B_{wn} C_{τ1} to C_{τn}, and C_{w1} to C_{wn} to be referred at time span S2 are transferred in parallel and simultaneously to the coefficient registers RA1, RA2, RB1 to RBn, and RC1 to RCn in sequence after the end of the referencing of the m number of initial coefficient data, and are updated. The period of transfer is set to be the same as the beam data computing period. In this way, successively storing reference data in the next time span immediately after referencing the lead of the last time division frame in each time span enables the updating for every time span without loss of the coefficients in the course of time divided multiple access. Further, after the updating and before the last time-division frame period, the same reference data is used in a cyclic manner. Thus, configuring that the coefficients to be used for computation is successively read out for every time span will obviate the need of providing a large storage space within the beamformer data computing unit 100 since a large amount of data which can be stored only in the external storage means EXTRM is successively transferred for every time span, enabling high-speed data reference. Further, by making the junction point of span in the time direction of the beamformer data to be common between the delay data and the weighing data, it is possible to configure the operations of reducing the junction point position information of spans, and referencing the coefficients of data computation from the storage means EXTRM, by a simple circuit.

In the computation processing, time divided multiple access frame length M=(number of channels CH)×(number of beams BM) is iterated by a predetermined number corresponding to different time values Tₛ. Since the computation processing is performed by time division, the coefficient sequencer SQLD stores m number of initial constant term D and the register values of the coefficient registers RA1, RA2, RB1 to RBn, and RC1 to RCn in accordance with the maximum time divided multiple access frame length. When M<m, coefficients corresponding to the number M of short time divided multiple access frames are iterated multiple times to be stored in m number of coefficient registers.

Stored values a11 to a1m and a21 to a2m, which are different for every channel and beam, of the coefficients A₁ and A₂ are stored in the coefficient registers RA1 and RA2. Stored values b11 to b1m, b21 to b2m, . . . , and bn1 to bnm of the coefficients B₁ to Bₙ are stored in the coefficient registers RB1 to RBn, and stored values c11 to c1m, c21 to c2m, . . . , and cn1 to cnm of the coefficients C₁ to Cₙ are stored in the coefficient registers RC1 to RCn.

As for the intra-span time Tₛ within the time span computed by the time divided multiple access sequencer SQGN, Tₛ² is computed by multiplication means MPYTT. The output of the multiplication means MPYTT is subjected to multiplication with the reference output (any of a11 to a1m) of the coefficient register RA1 by the subsequent multiplication means MPYA1, and A1TT which is A₁·Tₛ² computation term is outputted. At the multiplication means MPYA2, the product between the reference output of the coefficient register RA2 and Tₛ is computed and A2T which is the A₂·Tₛ computation term is outputted.

Further, the intra-span time Tₛ is subjected to multiplication with values respectively referenced from the stored values p11 to p1m, p21 to p2m, . . . , and pn1 to pnm of the parameter variable registers RP1, RP2, . . . , and RPn of P1 to Pn by the multiplication means MPYP1 to MPYPn, and P1·Tₛ, P2·Tₛ, . . . , and Pn·Tₛ computation terms are computed. The output of multiplication means MPYP1 to MPYPn is subjected to multiplication with the reference output of the coefficient registers RB1 to RBn by the multiplication means MPYB1 to MPYBn, and the output BP1, BP2, . . . , BPn of B1·P1·Tₛ, B2·P2·Tₛ, . . . , and Bn·Pn·Tₛ computation terms are outputted.

At the multiplication means MPYC1 to MPYCn, the product between reference outputs of the stored values c11 to c1m, c21 to c2m, . . . , and cn1 to cnm of the coefficient registers RC1 to RCn and values respectively referenced from the parameter variable registers RP1, RP2, . . . , and RPn, and the outputs CP1, CP2, . . . , and CPn of C₁·P₁, C₂·P₂·T_{S}, . . . , and Cn·Pn computation terms are outputted.

Computation outputs A1TTA2T, BP1 to BPn, and CP1 to CPn are summed at a summer SUM to provide an input to an accumulator ACUM. In the last time-division frame period in a time span after time span S1, the output of the summer SUM serves as the input to the accumulator ACUM, and is updated so as to be referenced as the initial value of the subsequent time spans. The selection of the initial value data by a selector SEL1 is performed by an instruction output LD of the time divided multiple access sequencer SQGN.

The receiving time TA from the start of beamformer data computation, calculated at the time divided multiple access sequencer SQGN is successively compared with reference values ap1 to apm from an aperture masking-time register RAP by a comparator CMP, and an instruction DG is outputted. At the selector SEL2, if the receiving time TA is not more than the time length ap1 to apm to disable the beamformer data computation output, the beamformer data computation output DAT is a disabled value 0, and if it exceeds the time length, the output of the accumulator ACUM will become the output of the beamformer data computation output DAT. In this way, by including the aperture masking-time register RAP for storing reference values ap1 to apm, and the selector SEL2 for making the computation result of the beamformer data a disabled value 0 according to the reference values ap1 to apm, it is possible to highly accurately obtain an output other than 0 from a partway time of time span in the first time span of beamformer data computation. If there is no such means available, when a beamformer data computation output, which is discontinuously started from a partway time of time span as the time function, is needed, a time-wise function of at most 2nd degree needs to be used for approximation, the initial approximate computation accuracy may not be sufficiently attained. According to this configuration, as to the data before a partway time of time span, the need of considering regularizing conditions in the piecewise approximation in time is obviated, and it becomes possible to make a first time span to be long thereby reducing the data amount.

The span size register RSPN, the time register RAP, the beam-channel designation register RADR, and the parameter variable registers RP1 to RPn are set by being written by the processor TCPU from the bus PA, and are referred by the reference address bus RA of the time divided multiple access sequencer SQGN during time division computation. The coefficient registers RA1, RA2, RB1 to RBn, and RC1 to RCn and the accumulating register RACM are set by being written by the coefficient sequencer SQLD from the bus SA, and are referenced through the reference address bus RA of the time divided multiple access sequencer SQGN.

The computation data formats of multiplication means MPYTT, MPYA1, MPYA2, MPYP1 to MPYPn, MPYB1 to MPYBn, and MPYC1 to MPYCn, and the summer SUM, the accumulator ACUM and the storage data formats of the coefficient registers RA1, RA2, RB1 to RBn, and RC1 to RCn, and the accumulating register RACM may be either an integer format or a floating point format. Further, the computation of these multiplication, addition, and summation is performed concurrently for v_{τ} and v_{w} independently.

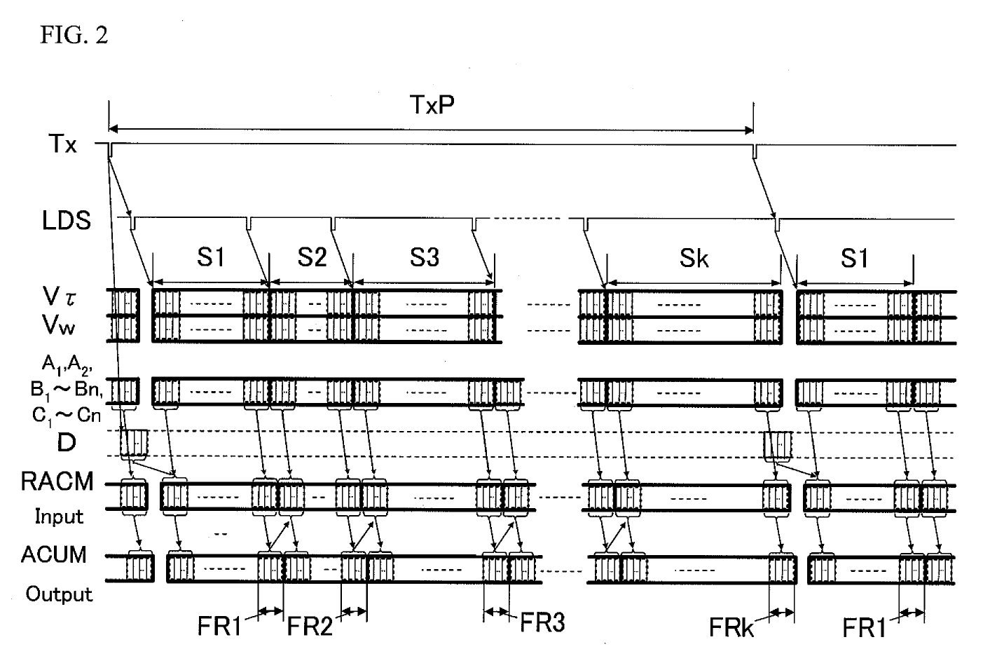

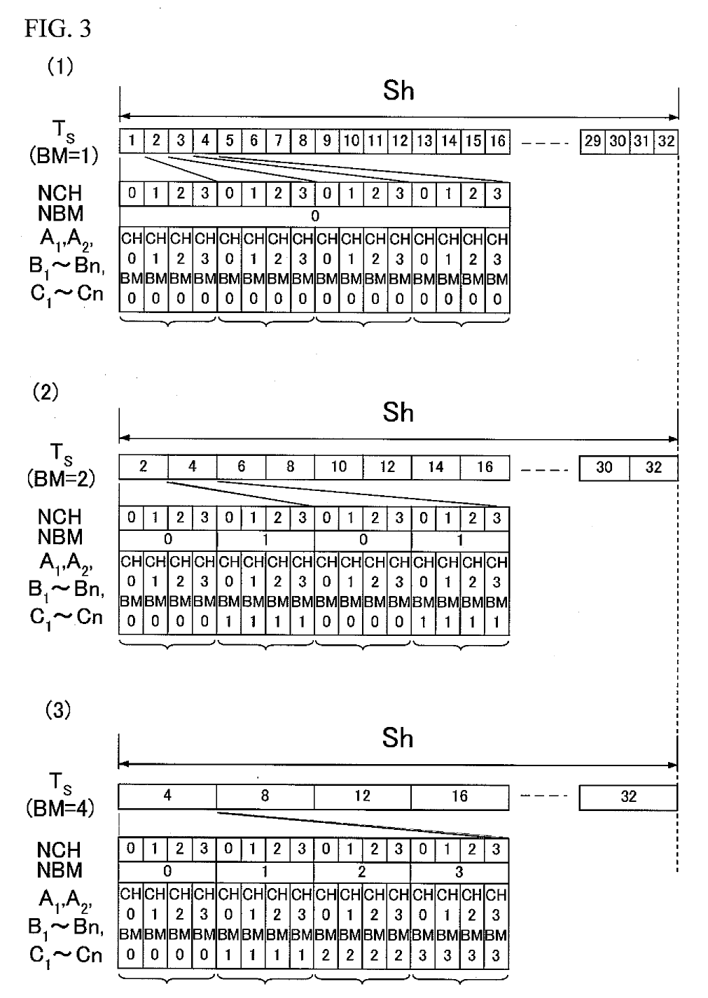

Next, the time divided multiple access of the processing of the beamformer data computing unit 100 of FIG. 1 will be described referring to FIGS. 2 and 3. The transmit synchronize reference signal Tx in FIG. 2 is a control signal for determining the reference time for transmission operation. During the transmit interval TxP, the time division operation of the beamformer data computing unit 100 is iterated.

The delay data τ and the weighing data w, which are the beamformer data, are functions which continuously change with time, and are computed with approximate functions v_{τ} and v_{w} for each time span which is divided into time spans S1 to Sk according to a time span load signal LDS. The time span load signal LDS is an internal instruction of the time divided multiple access sequencer SQGN of FIG. 1 and generates time segmentation positions according to the contents referenced from the span size register RSPN.

The initial constant term D stores initial values from the accumulating register RACM input according to the signal which is separately derived in synchronous manner from the transmit synchronize reference signal Tx. Further, coefficients A_{τ1}, A_{w1}, A_{τ2}, A_{w2}, B_{τ1} to B_{τn} B_{w1} to B_{wn}, C_{τ1} to C_{τn}, and C_{w1} to C_{wn} which are necessary for the computation coefficient in the first span S1 are similarly loaded depending on the transmit synchronize reference signal Tx. Reading out of reference is performed from the accumulating register RACM, and computation which concurrently uses the initial values D and the coefficients A_{τ1}, A_{w1}, A_{τ2}, A_{w2}, B_{τ1} to B_{τn}, B_{w1} to B_{wn}, C_{τ1} to C_{τn}, and C_{w1} to C_{wn}, which are necessary for the computation coefficients in the first span S1, is performed. At the last time division frame period FR1 of the time span S1, the output of the accumulator ACUM becomes the input to the accumulating register RACM and the content is updated. Thereafter, at the last time-division frame sections FR2 to FRk of the time spans S2 to S(k−1), the output of the accumulator ACUM becomes an updated input to the accumulating register RACM.

The time-division frame at the h-th time span Sh will be described in FIGS. 3(1), 3(2), and 3(3). The figures show the case in which the maximum number of beams BMmax=4, the number of channels CH=4, and the maximum length of time divided multiple access frame m=16. In FIG. 3(1), BM=1 and the time divided multiple access frame length M=4, and therefore m/M=4 and the coefficient group A_{τ1}, A_{w1}, A_{τ2}, A_{w2}, B_{τ1} to B_{τn}, B_{w1} to B_{wn}, C_{τ1} to C_{τn}, and C_{w1} to C_{wn} is iterated four times in the same sequence to be stored. Using a channel number NCH and a beam number NBM, that is equivalent with the four-time repetition of {CH0BM0, CH1BM0, CH2BM0, CH3BM0}. In the meantime, the intra-span time Tₛ is constant during {CH0BM0, CH1BM0, CH2BM0, CH3BM0}, and T_{S}=1, 2, 3, 4, 5, . . . every time the repetition is performed. In FIG. 3(2), BM=2 and the time divided multiple access frame length M=8, and therefore m/M=2 and the coefficient group A_{τ1}, A_{w1}, A_{τ2}, A_{w2}, B_{τ1} to B_{τn} B_{w1} to B_{wn}, C_{τ1} to C_{τn}, and C_{w1} to C_{wn} is iterated two times in the same sequence to be stored. Using the channel number NCH and the beam number NBM, that is equivalent with a two-time repetition of {CH0BM0, CH1BM0, CH2BM0, CH3BM0, CH0BM1, CH1BM1, CH2BM1, CH3BM1}. In the meantime, the intra-span time Tₛ is constant during {CH0BM0, CH1BM0, CH2BM0, CH3BM0, CH0BM1, CH1BM1, CH2BM1, CH3BM1}, and time is twice compared with the case of FIG. 3(1) and Tₛ=2, 4, 6, . . . every time the repetition is performed. In FIG. 3(3), BM=4 and the time divided multiple access frame length M=16, and therefore m=M and the coefficient group A_{τ1}, A_{w1}, A_{τ2}, A_{w2}, B_{τ1} to B_{τn}, B_{w1} to B_{wn}, C_{τ1} to C_{τn}, and C_{w1} to C_{wn} of a total length of m is stored. Using channel number NCH and beam number NBM, that will be {CH0BM0, CH1BM0, CH2BM0, CH3BM0, CH0BM1, CH1BM1, CH2BM1, CH3BM1, CH0BM2, CH1BM2, CH2BM2, CH3BM2, CH0BM3, CH1BM3, CH2BM3, CH3BM3}. In the meantime, the intra-span time Tₛ is constant, and compared with the case of FIG. 3(1), time is four times and Tₛ=4, 8, 12, . . . every time the repetition is performed.

As it is clearly seen from FIG. 3, using integer J and the size of span h, spnh=128, Tₛ is calculated by Tₛ=J×BM, (J=1, 2, 3, . . . , spnh/CH/BM). Since Ts becomes the same at the end of span h, the stored value of the accumulating register RACM remains the same even if the value of BM changes. As a result of this, even when change occurs such as BM=1, 2, 4, the time increment and initial values of Tₛ will change, and the for example beamformer data at NBM=0 can utilize {CH0BM0, CH1BM0, CH2BM0, CH3BM0} as the common data.

Thus, by making the time span to be processed to be common for every predetermined number of channel processes of beamformer, or for every predetermined beamforming condition, it is possible to perform computation by efficient time divided multiple access. In the current technology, it is possible to arrange that the computation frequency of the beamformer data computing unit 100 is higher than the read-out operation frequency of an implementation device of the external storage means EXTRM, and also it is possible to achieve a high packaging density by time divided multiple access.

Further, by making the above described time span size to be a common multiple of a predetermined number of channel processing or the number of beam processing, it is possible to perform computation without undesired processing stand-by time. Further, by changing the “initial value” or “change amount (increment etc.)” to be given to the generating computation of the time function Ts of each span for every predetermined number of channel processing, or every predetermined beamforming condition in the time span, and by performing the computation to generate a time variable Tₛ which will have the same value as the time variable Tₛ in the final computation of the span, it is possible to achieve the same beamformer output even when the output period of the channel and beam processing differ due to a different time division operation. As a result of this, since there will be no need of storing different beamformer data for every time-division operation condition in the external storage means EXTRM, the storage volume can be reduced. Further, when only part of the beamformer data is stored in the storage means EXTRM and the stored content of the storage means EXTRM is updated by transfer when the channel and beam processing setting are changed, it is possible to reduce the standby time due to transfer time until the start of imaging.

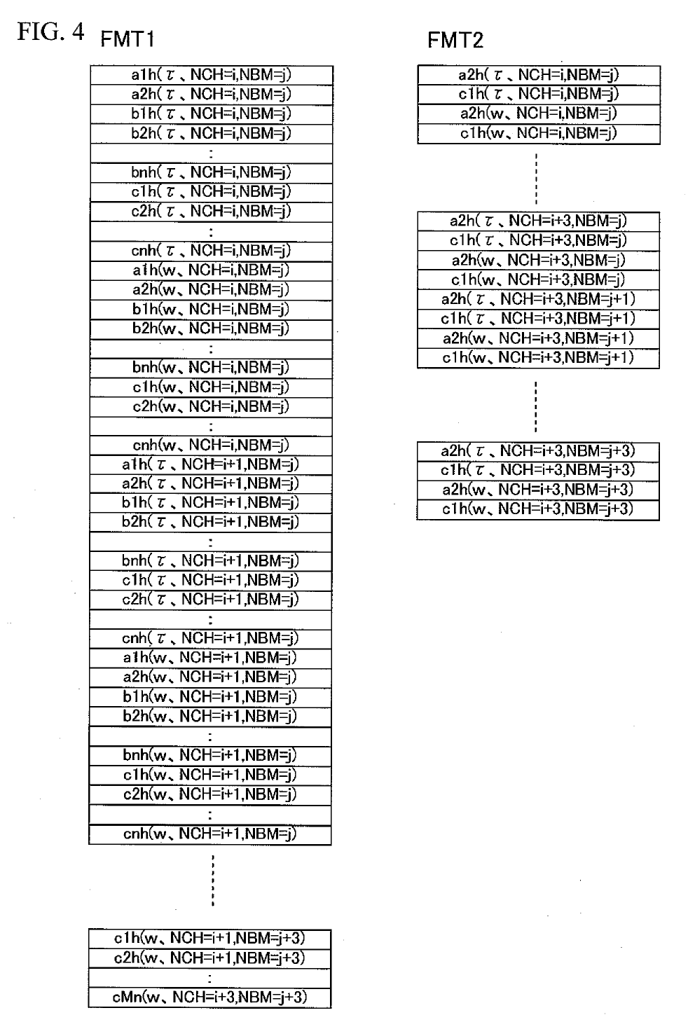

Next, a variable configuration example of the beamformer data format to be stored in the storage means EXTRM of FIG. 1 will be described in FMT1 and FMT2 of FIG. 4. FMT1 and FMT2 show the data format in the time span h for 4 channels of channel number NCH=i to i+3 and for 4 beams of beam number NBM=j to j+3. FMT1 shows a configuration of a longest format and FMT2 shows a configuration in which the kinds of coefficients are minimized. Other than these, although the initial value D etc. may be the input of beamformer data computing unit, those have a small data amount and therefore will not significantly change the volume and transfer speed of the storage means EXTRM.

In these data formats, the coefficient sequencer SQLD of FIG. 2 stores coefficients A_{τ1}, A_{w1}, A_{τ2}, A_{w2}, B_{τ1} to B_{τn}, B_{τ1} to B_{τn}, C_{τ1} to C_{τn}, and C_{w1} to C_{wn} into the coefficient registers RA1, RA2, RB1 to RBn, and RC1 to RCn corresponding to a plurality of beamformer data formats. The output of the coefficient sequencer SQLD is arranged such that a coefficient which is not included in the beamformer data format has a stored value of 0, and in a sum of product computation in a time span, the corresponding sum of product computation terms are neglected. It is noted that the data of FMT1 and FMT2 is transferred in an appropriately packed state in accordance with the bit width of the data bus, etc. of the storage means EXTRM, and the format of each coefficient will not be limited to the same bit width. The coefficient may be either in an integral format or a floating point format. By adopting a configuration in which a read operation to omit the read operation of the initial value and coefficients other than the coefficients of the time-wise linear function is selectable, it is possible to reduce the data transfer time from the storage means EXTRM and thereby transfer data corresponding to a large number of beams and channels even in a short time span.

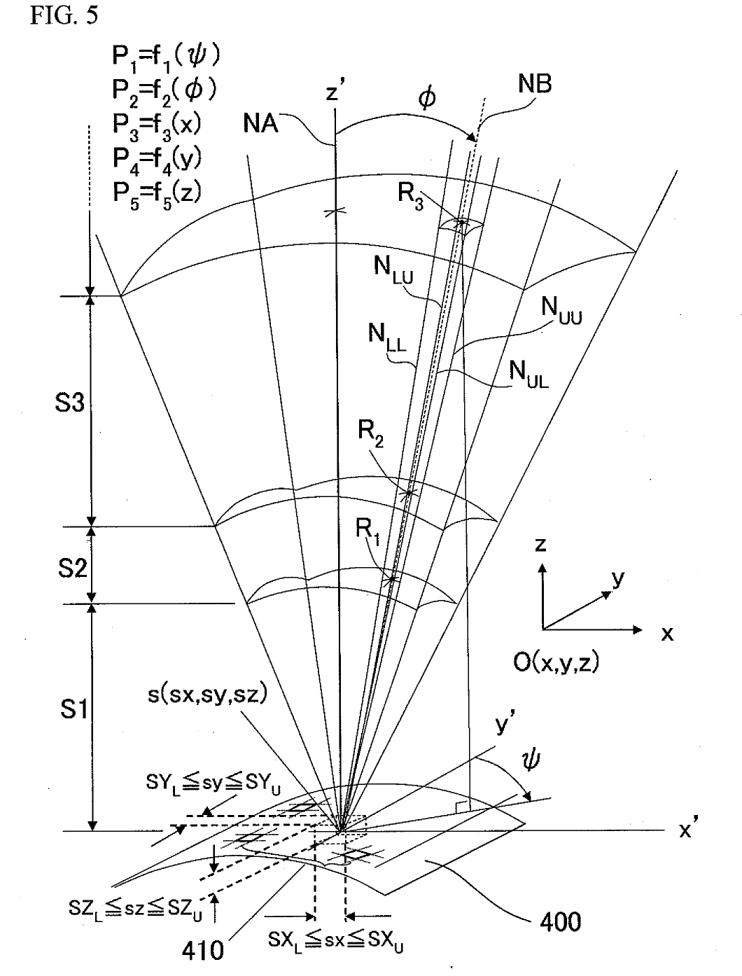

Next, for the purpose of imaging in a 3-dimensional space, there is shown in FIG. 5 an example in which parameters P₁ to P₅ of the parameters P₁ to Pn are brought into correspondence with beam steering direction angles ψ and φ, and the reference point coordinates s(sx, sy, sz) of the beam sound axis respectively in the processing of the beamformer data computing unit 100. Transmit and receive transducer elements 410 of a probe, which is not generally shown, for performing the transmission/reception of ultrasound are disposed to be separated at least two-dimensionally in space to realize a desired directivity property. Not all the elements of the transmit and receive transducer elements 410 need to have the same shape, and they may not be arranged in a lattice form. A plurality of input channel signals of the receiving beamformer 120 are formed independently from individual elements of the transmit and receive transducer elements 410 or by performing some signal processing on some of the elements to form a single input signal. A reference axis NA is defined by a coordinate system O(x, y, z) so as to be normal direction at the coordinate s(sx, sy, sz) in the acoustic output plane of the probe 400. The acoustical beamforming direction NB is uniquely determined by a steering direction angle (ψ,φ) in the coordinate system (x′, y′, z′) with the reference axis NA being the z′-axis. The parameters P₁ to P₅ in the beamformer data computing unit 100 are uniquely determined by using appropriate functions f₁ to f₅ as P₁=f₁(ψ), P₂=f₂(φ), P₃=f₃(sx), P₄=f₄(sy), and P₅=f₅(sz).

Under those conditions are set time spans S1, S2, S3, . . . , and Sk which are determined by the length interval formed by points R1, R2 . . . , Rk on the beam axis NB with s(sx, sy, sz) being the origin, and the sound velocity Vc. The beam steering direction angle (ψ,φ) can be approximately computed by defining an ideal beamformer data function v′ (Ts, P₁, P₂, P₃, P₄, P₅) of the delay data τ or the weighing data w in ψ_{U}≦ψ≦ψ_{L} and φ_{U}≦φ≦φ_{L} which is a solid angle span surrounded by the lines N_{LU}, N_{LL}, N_{UU}, N_{UL} from the origin s(sx, sy, sz) and in SX_{U}≦sx≦SX_{L}, SY_{U}≦sy≦SY_{L}, and SZ_{U}≦sz≦SZ_{L} of s(sx, sy, sz), and dividing this into time spans S1 to Sk to determine an approximate function of the beamformer data at a desired accuracy thereby determining coefficients. Thus, it is possible to divide the beamformer data for 3-dimensional imaging, in which five variables other than time are included, into groups of multiple spans which are represented by approximate functions with intra-span time T_{S} and parameters P₁ to P₅, and to calculate it by approximation under the constraint of achieving a desired accuracy. It is noted that the method to determine the acoustical beamforming direction NB is not limited to the method of determining the reference of angle of FIG. 5, and the forming direction may be specified in various ways; however, it remains true that the forming direction can be uniquely specified by three coordinates of two angles and an aperture position. By such an approximation, the necessary storage volume of the storage means EXTRM can be significantly reduced.

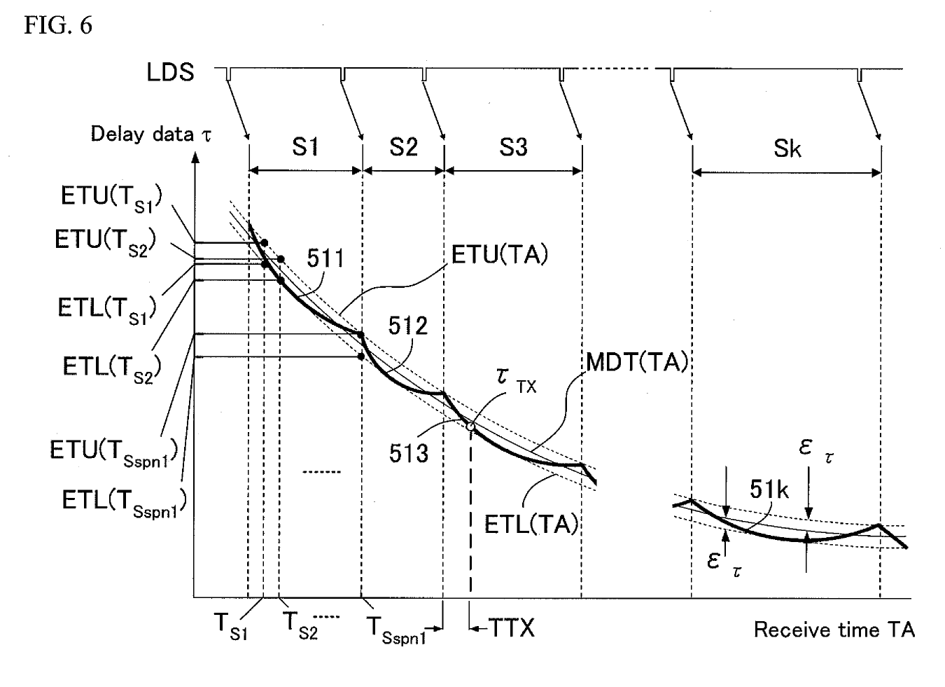

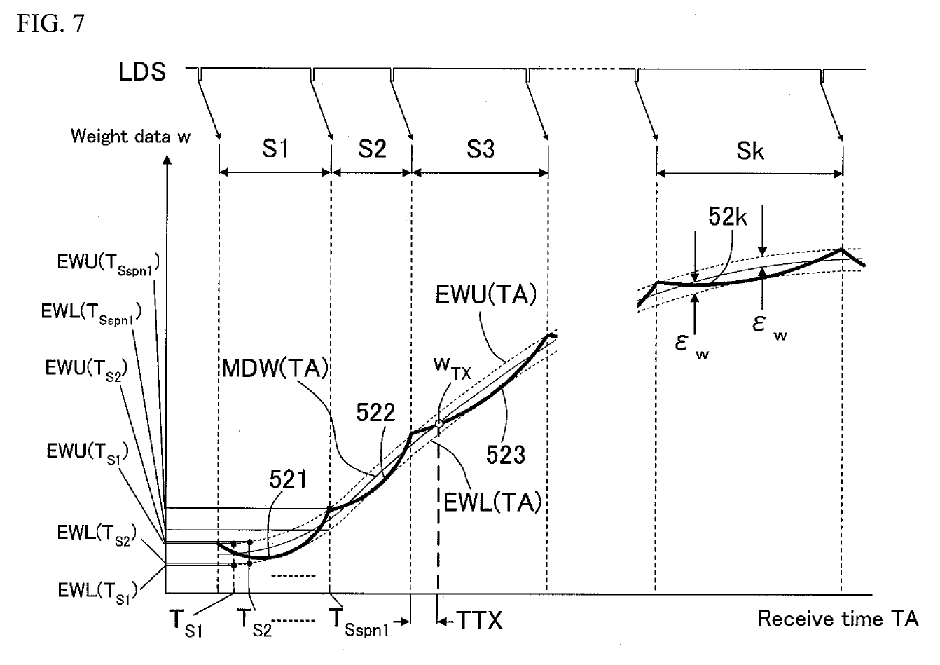

Next, regarding the coefficient group A_{τ1}, A_{w1}, A_{τ2}, A_{w2}, B_{τ1} to B_{τn}, B_{w1} to B_{wn}, C_{τ1} to C_{τn}, and C_{w1} to C_{wn} and the initial values D_{τ} and D_{w} to be provided to the beamformer data computing unit 100 of the present invention, an optimization method utilizing the linear programming calculation will be described with reference to FIGS. 6 and 7.

First, according to the design requirement of the beamformer, segmentation is performed on the receiving time TA, the channel number NCH, the beam number NBM, the parameter values NP1 to NPn of the parameters P₁ to Pn. There are determined k number of time segments S1 to Sk for the receiving time TA; kc number of channel segments CHS1 to CHSkc for the channel number NCH; kb number of beam segments BMS1 to BMSkb for the beam number NBM; and respectively k1 to kn number of beam segment groups P1S1 to P1Sk1, P2S1 to P2Sk2, . . . , PnS1 to PnSkn for the parameters P₁ to Pn. In accordance with the operation of the beamformer data computing unit 100, the beam segments BMS1 to BMSkb are grouped together for at least every BMmax, which is the maximum number of time-division beams. Further, when the same time segment is used in common for all the beams, the segment becomes a single segment, that is, kb=1. It is most efficient to group together the channel segments CHS1 to CHSkc in the number of time-division channels CH. The parameters P₁ to Pn can also be assigned as parameters relating to the beamformer data, for example, one or two angles relating to the deflection direction of the beam, one to three coordinates relating to the position specification of the transmitting/receiving aperture, the sound velocity, the transmitting/receiving frequency, temperature, imaging site, control input parameters as an adaptive beamformer, and thus can be segmented within the range of standard values of the parameters.

Next, channel segment CHSkcs is selected from channel segments CHS1 to CHSkc; beam segment BMSkbs from beam segments BMS1 to BMSkb; and parameter segments P1Sk1s to P1Skns from parameter segment group P1S1 to P1Sk1, P2S1 to P2Sk2, . . . , PnS1 to PnSkn. The channel segment CHSkcs includes CH number of channel number values, the beam segment BMSkbs includes BMmax number of beam number values, and the parameter segments P1Sk1s to P1Skns respectively include P1SNk1s to P1SNkns number of parameter standard values. Selecting channel number value NCH from the channel segment CHSkcs, beam number value NBM from beam segment BMSkbs, and parameter standard values NP1Sk1s to NP1Skns from the parameter segments P1Sk1s to P1Skns, by numerical calculation with sufficient accuracy, a time function of reference beamformer data is created to calculate an ideal delay data function MDT(TA) of the delay time τ of FIG. 6 and an ideal weighing data function MDW(TA) of the weighting w of FIG. 7. In actual beamformer computation, an error of ±ε_{τ} where ε_{τ}>0 is set for the ideal delay data function MDT(TA) of the delay time τ, and an allowance of ±ε_{w} where ε_{w}>0 is set for the ideal weighing data function MDW(TA) of the weighting w. As a result of this, an allowable upper limit function ETU(TA) and allowable lower limit function ETL(TA) of the delay time τ, and an allowable upper limit function EWU(TA) and allowable lower limit function EWL(TA) of the weighting w can be defined.

ETU(TA)=MDT(TA)+ε_{τ}

ETL(TA)=MDT(TA)−ε_{τ}

EWU(TA)=MDW(TA)+ε_{w}

EWL(TA)=MDW(TA)−ε_{w}

These time functions are calculated at a period of intra-span time T_{S} discretely generated by the time divided multiple access sequencer SQGN of the beamformer data computing unit 100. The setting of boundary conditions for the linear programming will be illustrated on the first time span S1 with reference to FIGS. 6 and 7. For time points T_{S1}, T_{S2}, . . . , T_{Sspn1}, in the time span S1, boundary upper limit values ETU(T_{S1}), ETU(T_{S2}), ETU(T_{Sspn1}), EWU(T_{S1}), EWU(T_{S2}), and EWU(T_{Sspn1}), and boundary lower limit values ETL(T_{S1}), ETL(T_{S2}), ETL(T_{Sspn1}), EWL(T_{S1}), EWL(T_{S2}), and EWL(T_{Sspn1}) are set. A linear programming problem LP1 shown below is set in which the above described boundary constraint relationships are applied to the simultaneous combination of all the values in the channel segment CHSkcs, the beam segment BMSkbs, and the parameter segments P1Sk1s to P1Skns.

### Linear Programming Problem LP1:

Simultaneous inequalities are established for

1) All the channel number values in the channel segment CHSkcs,
2) All the beam number values in the beam segment BMSkbs, and
3) All the parameter standard values in the parameter segments P1Sk1s to P1Skns, with a_{τ1} and a_{τ2} being unknowns corresponding to coefficients A_{τ1} and A_{τ2} of the delay time τ, b_{τ1} to b_{τn} and c_{τ1} to c_{τn} being unknowns corresponding to the coefficients B_{τ1} to B_{τn} and C_{τ1} to C_{τn}, and d_{τ} being an unknown corresponding to initial value D_{τ}.

$$\begin{matrix}\{ \begin{matrix}{a}_{τ 1} {T}_{S 1}^{2} + {a}_{τ 2} {T}_{S 1} + {\sum }_{i = 1}^{n} ( \begin{matrix}{b}_{τ i} \cdot {T}_{S 1} \cdot \\ {p}_{i } + {c}_{τ i} \cdot {p}_{i}\end{matrix} ) + \\ {d}_{τ} \leq MDT ( {T}_{S 1} ) + {Q}_{τ} \\ {a}_{τ 1} {T}_{S 2}^{2} + {a}_{τ 2} {T}_{S 2} + {\sum }_{i = 1}^{n} ( \begin{matrix}{b}_{τ i} \cdot {T}_{S 2} \cdot \\ {p}_{i} + {c}_{τ i} \cdot {p}_{i}\end{matrix} ) + \\ {d}_{τ} \leq MDT ( {T}_{S 2} ) + {Q}_{τ} \\ ⋮ \\ {a}_{τ1 } {T}_{Sspn 1}^{2} + {a}_{τ 2} {T}_{Sspn 1} + {\sum }_{i = 1}^{n} ( \begin{matrix}{b}_{τ i} \cdot {T}_{Sspn 1} \cdot \\ {p}_{i} + {c}_{τ i} \cdot {p}_{i }\end{matrix} ) + \\ {d}_{τ} \leq MDT ( {T}_{Sspn 1} ) + {Q}_{τ}\end{matrix} & [ Expression 3 ] \\ \{ \begin{matrix}{a}_{τ 1} {T}_{S 1}^{2} + {a}_{τ 2} {T}_{S 1} + {\sum }_{i = 1}^{n} ( {b}_{τ i} \cdot {T}_{S 1} \cdot {p}_{i} + {c}_{τ i} \cdot {p}_{i } ) + \\ {d}_{τ} \geq MDT ( {T}_{S 1} ) - {Q}_{τ} \\ {a}_{τ 1 } {T}_{S 2}^{2} + {a}_{τ 2} {T}_{S 2} + {\sum }_{i = 1}^{n} ( {b}_{τ i} \cdot {T}_{S 2} \cdot {p}_{i} + {c}_{τ i} \cdot {p}_{i} ) + \\ {d}_{τ} \geq MDT ( {T}_{S 2} ) - {Q}_{τ} \\ ⋮ \\ {a}_{τ 1} {T}_{Sspn 1}^{2} + {a}_{τ 2} {T}_{Sspn 1} + {\sum }_{i = 1}^{n} ( {b}_{τ i} \cdot {T}_{Sspn 1} \cdot {p}_{i} + {c}_{τ i} \cdot {p}_{i} ) + \\ {d}_{τ} \geq MDT ( {T}_{Sspn 1} ) - {Q}_{τ}\end{matrix} & \\ {Q}_{τ} \leq {ɛ}_{τ} \\ {Q}_{τ} -> 0 & \end{matrix}$$

The number of simultaneous inequalities will be 2×spn1×CHSkcs×BMSkbs×P1Sk1s×P1Sk2s×, . . . , ×P1Skns+1, and optimization to minimize an objective function Q_{τ} is performed with a calculation algorithm such as the modified simplex algorithm.

When the linear programming problem LP1 has no solution, ε_{τ} is reset or the span size spn1 of S1 is shortened, and the solving solution is iterated. When the linear programming problem LP1 is solved, successively similar linear programming problem LP2 relating to the weighting will be determined.

### Linear Programming Problem LP2:

The following simultaneous inequalities are established for

1) All the channel number values in the channel segment CHSkcs,
2) All the beam number value in the beam segment BMSkbs, and
3) All the parameter standard values in the parameter segments P1Sk1s to P1Skns, with a_{w1} and a_{w2} being unknowns corresponding to coefficients A_{w1} and A_{w2} of the weighting w, b_{w1} to b_{wn} and c_{w1} to c_{wn} being unknowns corresponding to the coefficients B_{w1} to B_{wn} and C_{w1} to C_{wn}, and d_{w} being an unknown corresponding to initial value D_{w}.

$$\begin{matrix}\{ \begin{matrix}\begin{matrix}{a}_{w 1} {T}_{S 1}^{2} + {a}_{w 2} {T}_{S 1} + {\sum }_{i = 1}^{n} ( \begin{matrix}{b}_{wi} \cdot {T}_{S 1} \cdot \\ {p}_{i} + {c}_{wi} \cdot {p}_{i}\end{matrix} ) + \\ {d}_{w} \leq MDW ( {T}_{S 1} ) + {Q}_{w}\end{matrix} \\ {a}_{w 1} {T}_{S 2}^{2} + {a}_{w 2} {T}_{S 2} + {\sum }_{i = 1}^{n} ( \begin{matrix}{b}_{wi} \cdot {T}_{S 2} \cdot \\ {p}_{i} + {c}_{wi} \cdot {p}_{i}\end{matrix} ) + \\ {d}_{w} \leq MDW ( {T}_{S 2} ) + {Q}_{w} \\ ⋮ \\ {a}_{w 1} {T}_{Sspn 1}^{2} + {a}_{ w 2} {T}_{Sspn 1} + {\sum }_{i = 1}^{n} ( \begin{matrix}{b}_{w i} \cdot {T}_{Sspn 1} \cdot \\ {p}_{i} + {c}_{w i} \cdot {p}_{i}\end{matrix} ) + \\ {d}_{w} \leq MDW ( {T}_{Sspn 1} ) + {Q}_{w}\end{matrix} & [ Expression 4 ] \\ \{ \begin{matrix}\begin{matrix}{a}_{w 1} {T}_{S 1}^{2} + {a}_{w 2} {T}_{S1} + {\sum }_{i = 1}^{n} ( \begin{matrix}{b}_{wi} \cdot {T}_{S 1} \cdot {p}_{i} + \\ {c}_{wi} \cdot {p}_{i}\end{matrix} ) + \\ {d}_{w} \geq MDW ( {T}_{S1} ) + {Q}_{w}\end{matrix} \\ \begin{matrix}{a}_{w 1} {T}_{S 2 }^{2} + {a}_{w 2} {T}_{S 2} + {\sum }_{i = 1}^{n} ( \begin{matrix}{b}_{w i} \cdot {T}_{S 2} \cdot {p}_{i} + \\ {c}_{wi} \cdot {p}_{i}\end{matrix} ) + \\ {d}_{w} \geq MDW ( {T}_{S 2} ) - {Q}_{w} \\ ⋮ \\ {a}_{w 1} {T}_{Sspn 1}^{2} + {a}_{w 2} {T}_{Sspn 1} + {\sum }_{i = 1}^{n} ( \begin{matrix}{b}_{wi} \cdot {T}_{Sspn 1} \cdot {p}_{i} + \\ {c}_{wi} \cdot {p}_{i}\end{matrix} ) + \\ {d}_{w} \geq MDW ( {T}_{Sspn 1} ) - {Q}_{w}\end{matrix}\end{matrix} & \\ {Q}_{w} \leq {ɛ}_{w} \\ {Q}_{w} -> 0 & \end{matrix}$$

When the linear programming problem LP2 has no solution, ε_{w} is reset or the span size spn1 of S1 is shortened, and returning to the linear programming problem LP1, the solving solution is iterated. Until the linear programming problems LP1 and LP2 are simultaneously solved, the repetition is performed. When the span size spn1 of S1 is set to be short, the starting point of the following span of S2 is changed.

The solving solution of the above described linear programming problems LP1 and LP2 is iterated for all the time span Sk, and the coefficients which are unknowns are determined by successive repetitions such that the end point of the span Sk is included in the last time point in the range divided into time spans. In this way, optimum function curves 511 to 51k, and 521 to 52k in the spans are determined and beamformer data within a desired accuracy range is obtained.

Further, newly selecting segments from the channel segments CHS1 to CHSkc, the beam segments BMS1 to BMSkb, and the parameter segment groups P1S1 to P1Sk1, P2S1 to P2Sk2, . . . , and PnS1 to PnSkn, the linear programming problems LP1 and LP2 are iterated to determine the coefficients which are unknowns thus calculating the entire beamformer data.

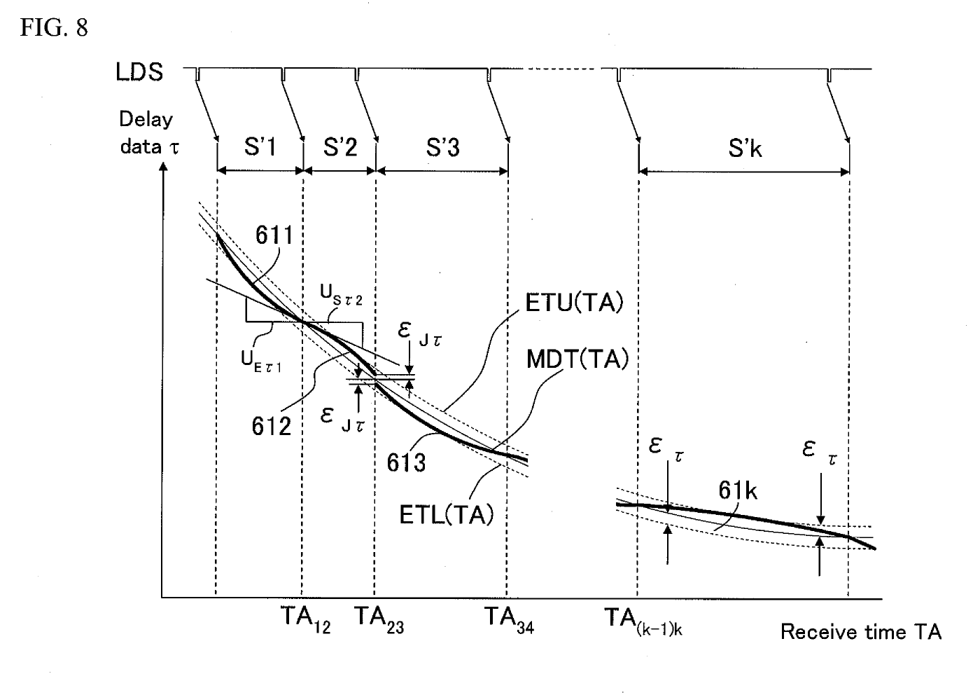

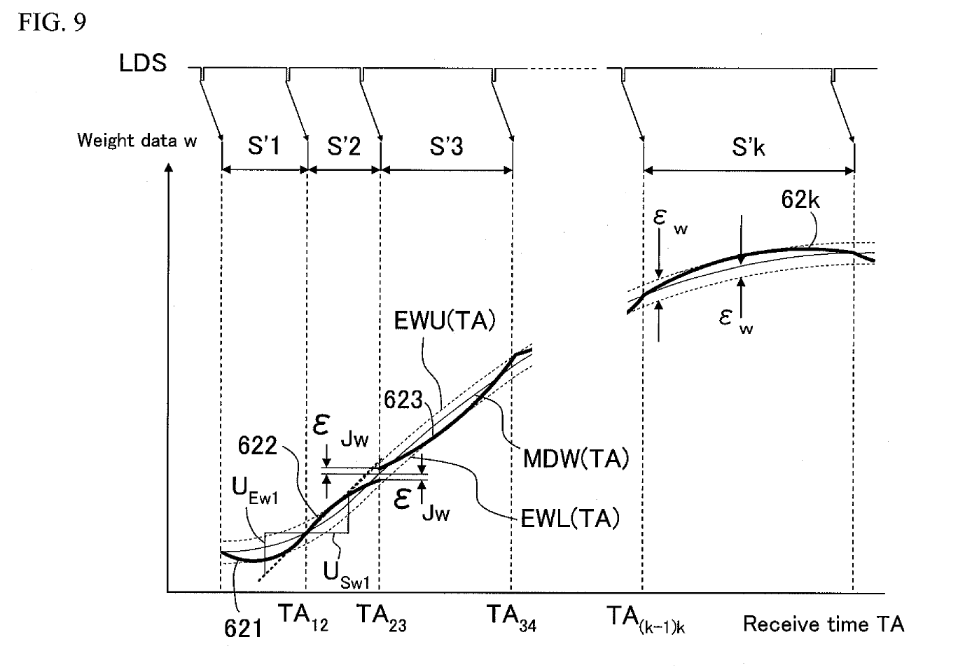

Next, a solving solution by linear programming, in which continuity regularizing condition of operation value at the connection point between time spans, and continuity regularizing condition of time-wise 1st partial differential coefficients are added, will be described with reference to FIGS. 8 and 9. FIGS. 8 and 9 show the results of optimization of the ideal delay data function MDT(TA) of the delay time τ of FIG. 6 and the ideal weighing data function MDW(TA) of the weighting w of FIG. 7 under the constraints of the continuity condition at connection point between time spans in addition to the limitation of deviation allowance. As with the cases of FIGS. 6 and 7, the receiving time TA is divided into time spans S′1 to S′k. Let spn′1 to spn′k be the span sizes of the time spans S′1 to S′k. Regarding the connecting time points TA₁₂ to TA₍ₖ₋₁₎ₖ of the spans, continuity regularizing conditions of the function values and the time-wise 1st partial differential coefficients are added. The continuity regularizing condition of function values is assumed to be the condition in which the errors of the ideal delay data function MDT(TA) of the delay time τ and the ideal weighing data function MDW(TA) of the weighting w at a time of a junction point between spans are constrained in the range of ±ε_{jτ} and ±ε_{Jw} with the errors being 0≦ε_{Jτ}≦ε_{τ} and 0≦ε_{Jw}≦ε_{w}.

Further, as for the continuity regularizing condition of the time-wise 1st partial differential coefficient as well, regularizing conditions at junction points of span are determined on time-wise 1st partial differential functions u_{τ}(Ts, P₁, P₂, . . . , Pn) and u_{w}(Ts, P₁, P₂, . . . , Pn) defined by the following equations.

$$\begin{matrix}{u}_{τ} ( Ts , {P}_{1} , {P}_{2} , … , {P}_{n} ) ≡ \frac{\partial {v}_{τ} ( Ts , {P}_{1} , {P}_{2} , … , {P}_{n} )}{\partial Ts} \\ = 2 {A}_{τ 1} {T}_{S} + {A}_{τ 2} + {\sum }_{i = 1}^{n} {B}_{τ i} \cdot {P}_{i}\end{matrix} \\ \begin{matrix}{u}_{w} ( Ts , {P}_{1 } , {P}_{2} , … , {P}_{n} ) ≡ \frac{\partial {v}_{w} ( Ts , {P}_{1} , {P}_{2} , … , {P}_{n} )}{\partial Ts} \\ = 2 {A}_{w 1} {T}_{S} + {A}_{w 2} + {\sum }_{i = 1}^{n} {B}_{wi} \cdot {P}_{i}\end{matrix} \qquad [ Expression 5 ]$$

Letting spn′h be the span size of h-th time span S′h, the values of the first partial derivative with respect to time U_{Eτh}=u_{τ}(Ts_{spn′h}, P₁, P₂, . . . , Pn) and U_{Ewh}=u_{w}(Ts_{spn′h}, P₁, P₂, . . . , Pn) at time point T_{S}=T_{Sppn′h} of the end point of intra-span time Tₛ are calculated. In the solving solution of the linear programming problem at the (h+1)th time span S′(h+1) where h≧1, setting is made such that a time point, at which Tₛ=0, prior to a lead time Tₛ₁ in the S′(h+1) span coincides with a time point T_{S}=T_{Sspn′h} of the end point of the h-th time span S′h.

The time-wise 1st differential coefficients U_{Sτh+1}=u_{τ}(0, P₁, P₂, . . . , Pn) and U_{Swh+1}=u_{w}(0, P₁, P₂, . . . , Pn) at Tₛ=0 in the time span S′(h+1) are obtained, the allowance of ε_{Dτ} where ε_{Dw}>0 for the absolute value of difference is defined, and regularizing conditions |U_{Sτh+1}−U_{Eτh+1}|≦ε_{Dτ} and |U_{Swh+1}−U_{Ewτh+1}|≦ε_{Dw} are given to solve solution. It is noted that FIGS. 8 and 9 illustrate allowable errors of ±ε_{Jτ} and ±ε_{Jw} for the position at which h=2 and junction point time is TA₂₃. Further, for the position at which the junction point time is TA₁₂, the continuity of the time-wise first partial differential function is illustrated.

Under new regularizing conditions, the following linear programming problem LP1d will be set.

### Linear Programming Problem LP1d:

The following simultaneous inequalities are established for

1) All the channel number values in the channel segment CHSkcs,
2) All the beam number value in the beam segment BMSkbs, and
3) All the parameter standard values in the parameter spans segments to P1Skns, with a_{τ1} and a_{τ2} being unknowns corresponding to coefficients A_{τ1} and A_{τ2} of the delay time τ, b_{τ1} to b_{τn} and c_{τ1} to c_{τn} being unknowns corresponding to the coefficients B_{τ1} to B_{τn} and C_{τ1} to C_{τn}, and d_{τ} being an unknown corresponding to initial value D_{τ}.

$$\begin{matrix}\{ \begin{matrix}{\sum }_{i = 1}^{n} {c}_{τ i} \cdot {p}_{i} + {D}_{τ} \leq MDT ( 0 ) + {ɛ}_{J τ} \\ {a}_{τ 1} {T}_{S 1}^{2} + {a}_{τ 2} {T}_{S 1} + {\sum }_{i = 1}^{n} ( {b}_{τ i} \cdot {T}_{S 1} \cdot {p}_{i} + {c}_{τ i} \cdot {p}_{i} ) + \\ {d}_{τ} \leq MDT ( {T}_{S 1} ) + {Q}_{τ} \\ {a}_{τ 1} {T}_{S 2}^{2} + {a}_{τ 2} {T}_{S 2} + {\sum }_{i = 1}^{n} ( {b}_{τ i} \cdot {T}_{S 2} \cdot {p}_{i} + {c}_{τ i} \cdot {p}_{i} ) + \\ {d}_{τ} \leq MDT ( {T}_{S 2} ) + {Q}_{τ} \\ ⋮⋮ \\ {a}_{τ1} {T}_{S ( spn 1 - 1 )}^{2} + {a}_{τ 2} {T}_{S ( spn 1 - 1 )} + {\sum }_{i = 1}^{n} ( \begin{matrix}{b}_{τ i} \cdot {T}_{S ( spn 1 - 1 )} \cdot \\ {p}_{i} + {c}_{τ i} \cdot {p}_{i}\end{matrix} ) + \\ {d}_{τ} \leq MDT ( {T}_{S ( spn 1 - 1 )} ) + {Q}_{τ} \\ {a}_{τ 1 } {T}_{Sspn 1}^{2} + {a}_{τ 2 } {T}_{Sspn 1 } + {\sum }_{i = 1}^{n} ( \begin{matrix}{b}_{τ i} \cdot {T}_{Sspn 1} \cdot \\ {p}_{i} + {c}_{τ i} \cdot {p}_{i}\end{matrix} ) + \\ {d}_{τ} \leq MDT ( {T}_{Sspn 1} ) + {ɛ}_{J τ}\end{matrix} & [ Expression 6 ] \\ \{ \begin{matrix}\begin{matrix}{\sum }_{i = 1}^{n} {c}_{τ i} \cdot {p}_{i} + {d}_{τ} \geq MDT ( 0 ) - {ɛ}_{J τ} \\ {a}_{τ 1} {T}_{S 1}^{2} + {a}_{τ 2 } {T}_{ S 1 } + {\sum }_{i = 1}^{n} ( {b}_{τ i} \cdot {T}_{S 1} \cdot {p}_{i} + {c}_{τ i} \cdot {p}_{i } ) +\end{matrix} \\ {d}_{τ} \geq MDT ( {T}_{S 1} ) - {Q}_{τ} \\ {a}_{τ 1} {T}_{S 2}^{2} + {a}_{τ 2} {T}_{S 2} + {\sum }_{i = 1}^{n} ( {b}_{τ i} \cdot {T}_{S 2 } \cdot {p}_{i} + {c}_{τ i} \cdot {p}_{i} ) + \\ {d}_{τ} \geq MDT ( {T}_{S 2} ) - {Q}_{τ} \\ ⋮ \\ {a}_{τ 1} {T}_{S ( spn 1 - 1 )}^{2} + {a}_{τ 2} {T}_{S ( spn 1 - 1 )} + {\sum }_{i = 1}^{n} ( \begin{matrix}{b}_{τ i} \cdot {T}_{S ( spn 1 - 1 )} \cdot \\ {p}_{i} + {c}_{τ i} \cdot {p}_{i }\end{matrix} ) + \\ {d}_{τ} \geq MDT ( {T}_{S ( spn 1 - 1 )} ) - {Q}_{τ} \\ {a}_{τ 1} {T}_{Sspn 1}^{2} + {a}_{τ 2} {T}_{Sspn 1} + {\sum }_{i = 1}^{n} ( {b}_{τ i} \cdot {T}_{Sspn 1} \cdot {p}_{i } + {c}_{τ i} \cdot {p}_{i} ) + \\ {d}_{τ} \geq MDT ( {T}_{Sspn 1} ) - {ɛ}_{J τ}\end{matrix} & \\ \{ \begin{matrix}{a}_{τ 2} + {\sum }_{i = 1}^{n} {b}_{τ i} \cdot {p}_{i} - {U}_{E τ h} \leq {ɛ}_{D τ} \\ {a}_{τ 2} + {\sum }_{i = 1}^{n} {b}_{τ i } \cdot {p}_{i} - {U}_{E τ h} \geq - {ɛ}_{D τ}\end{matrix} & \\ {Q}_{τ} \leq {ɛ}_{τ} \\ {Q}_{τ} -> 0 & \end{matrix}$$

An optimization to minimize an objective function Q_{τ} is performed with a calculation algorithm such as the modified simplex algorithm. When the linear programming problem LP1d has no solution, ε_{τ}, ε_{Jτ}, and ε_{Dτ} are reset or the span size spn′h of S′h is shortened, and the solving solution is iterated. When the linear programming problem LP1d is solved, the solution of a similar linear programming problem LP2d relating to the weighting w is determined.

### Linear Programming Problem LP2:

The following simultaneous inequalities are established for

1) All the channel number values in the channel segment CHSkcs,
2) All the beam number values in the beam segment BMSkbs, and
3) All the parameter standard values in the parameter segments P1Sk1s to P1Skns,
with a_{w1} and a_{w2} being unknowns corresponding to coefficients A_{w1} and A_{w2} of the weighting w, b_{w1} to b_{wn} and c_{w1} to c_{wn} being unknowns corresponding to the coefficients B_{w1} to B_{wn} and C_{w1} to C_{wn}, and d_{w} being an unknown corresponding to initial value D_{w}.

$$\begin{matrix}\{ \begin{matrix}{\sum }_{i = 1}^{n} {c}_{wi} \cdot {p}_{i} + {D}_{w} \leq MDW ( {T}_{S 1} ) + {ɛ}_{Jw} \\ {a}_{w 1} {T}_{S 1}^{2} + {a}_{w 2} {T}_{S 1} + {\sum }_{i = 1}^{n} ( {b}_{wi} \cdot {T}_{S 1} \cdot {p}_{i} + {c}_{wi} \cdot {p}_{i } ) + \\ {d}_{w} \leq MDW ( {T}_{S 1} ) + {Q}_{w} \\ {a}_{w 1} {T}_{S 2}^{2} + {a}_{w 2} {T}_{S 2} + {\sum }_{i = 1}^{n} ( {b}_{wi} \cdot {T}_{S 2} \cdot {p}_{i} + {c}_{wi} \cdot {p}_{i} ) + \\ {d}_{w} \leq MDW ( {T}_{S 2} ) + {Q}_{w} \\ ⋮ \\ {a}_{w 1} {T}_{S ( spn 1 - 1 )}^{2} + {a}_{w 2} {T}_{S ( spn 1 - 1 )} + {\sum }_{i = 1}^{n} ( \begin{matrix}{b}_{wi} \cdot {T}_{S ( spn 1 - 1 )} \cdot \\ {p}_{i} + {c}_{wi} \cdot {p}_{i}\end{matrix} ) + \\ {d}_{w} \leq MDW ( {T}_{S ( spn 1 - 1 )} ) + {Q}_{w} \\ {a}_{w 1} {T}_{Sspn 1}^{2} + {a}_{w 2} {T}_{Sspn 1} + {\sum }_{i = 1}^{n} ( \begin{matrix}{b}_{w 1} \cdot {T}_{Sspn 1} \cdot \\ {p}_{i } + {c}_{wi} \cdot {p}_{i}\end{matrix} ) + \\ {d}_{w} \leq MDW ( {T}_{Sspn 1} ) + {ɛ}_{Jw}\end{matrix} & [ Expression 7 ] \\ \{ \begin{matrix}{\sum }_{i = 1}^{n} {c}_{wi} \cdot {p}_{i} + {d}_{w} \geq MDW ( {T}_{S 1} ) - {ɛ}_{J w} \\ {a}_{w 1} {T}_{S 1}^{2} + {a}_{w 2} {T}_{S 1} + {\sum }_{i = 1}^{n} ( {b}_{w i} \cdot {T}_{S 1} \cdot {p}_{i} + {c}_{wi} \cdot {p}_{i} ) + \\ {d}_{w} \geq MDW ( {T}_{S 1} ) - {Q}_{w} \\ {a}_{w 1} {T}_{S 2}^{2} + {a}_{w 2} {T}_{S 2} + {\sum }_{i = 1}^{n} ( {b}_{wi} \cdot {T}_{S 2} \cdot {p}_{i } + {c}_{wi} \cdot {p}_{i} ) + \\ {d}_{w} \geq MDW ( {T}_{S 2} ) - {Q}_{w} \\ ⋮ \\ {a}_{w 1} {T}_{S ( spn 1 - 1 )}^{2} + {a}_{w 2} {T}_{S ( spn 1 - 1 )} + {\sum }_{i = 1}^{n} ( \begin{matrix}{b}_{wi} \cdot {T}_{S ( spn 1 - 1 )} \cdot \\ {p}_{i} + {c}_{wi} \cdot {p}_{i}\end{matrix} ) + \\ {d}_{w} \geq MDW ( {T}_{S ( spn 1 - 1 )} ) - {Q}_{w} \\ \begin{matrix}{a}_{w 1} {T}_{Sspn 1}^{2} + {a}_{w 2} {T}_{Sspn 1} + {\sum }_{i = 1}^{n} ( \begin{matrix}{b}_{wi} \cdot {T}_{Sspn 1} \cdot \\ {p}_{i} + {c}_{wi} \cdot {p}_{i}\end{matrix} ) + \\ {d}_{w} \geq MDW ( {T}_{Sspn 1} ) - {{ɛ}_{J}}_{w}\end{matrix}\end{matrix} & \\ \{ \begin{matrix}{a}_{w 2} + {\sum }_{i = 1}^{n} {b}_{wi} \cdot {p}_{i} - {U}_{Ewh} \leq {ɛ}_{Dw} \\ {a}_{w 2} + {\sum }_{i = 1}^{n} {b}_{wi} \cdot {p}_{i} - {U}_{Ewh} \geq - {ɛ}_{D w}\end{matrix} & \\ {Q}_{w} \leq {ɛ}_{w} \\ {Q}_{w} -> 0 & \end{matrix}$$

When the linear programming problem LP2d has no solution, ε_{w}, ε_{Jw}, and ε_{Dw} are reset or the span size spn′h of S′h is shortened, and returning to the linear programming problem LP1d, the solving solution is iterated. The repetition is continued until the linear programming problems LP1d and LP2d are simultaneously solved. When the span size spn′h of S′h is set to be short, the starting point of the following span of S′(h+1) is changed.

The solving solution of the above described linear programming problems LP1d and LP2d is iterated for all the time spans S′1 to S′k, and the coefficients which are unknowns are determined by successive repetitions such that the end point of the span Sk is included in the last time point in the range divided into time spans. In this way, optimum function curves 611 to 61k and 621 to 62k in the spans are determined and beamformer data of higher continuity within a desired accuracy range is obtained.

Further, newly selecting segments from the channel segments CHS1 to CHSkc, the beam segments BMS1 to BMSkb, and the parameter segment groups P1S1 to P1Sk1, P2S1 to P2Sk2, . . . , and PnS1 to PnSkn, and the linear programming problems LP1d and LP2d are iterated to determine the coefficients which are unknowns thus calculating the entire beamformer data. Although in this example, the regularizing condition of error is supposed to be constant excepting junction points of time spans, it is needless to say that different regularizing condition may be given for every time point in the time span and for every time span. Although the calculation algorithm and regularizing conditions of the linear programming are not limited to the above described examples, they are characterized in that at least approximate function computing coefficients are determined by the linear programming from multiple regularizing condition inequalities which are divided into multiple time spans. A specific implementation can be realized by implementing this linear programming algorithm as a computing program for the TCPU of FIG. 11, and storing the result as function computation coefficient terms into storage means EXTRM. By determining the approximate function computation coefficient terms by a linear programming, it is possible to optimize the beamformer data within a desired accuracy range for a parameter or multiple parameters which are not necessarily continuous, enabling extremely effective implementation of beamformer data.

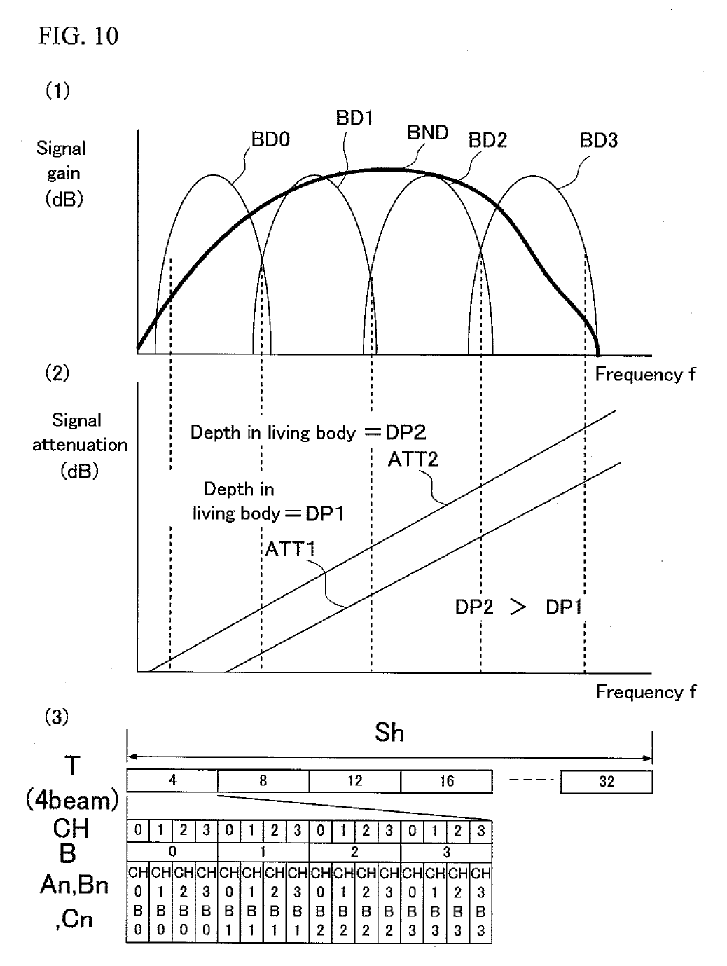

Next, description will be made on beamformer data in which signal frequency is divided for every band. FIG. 10(1), in which the axis of abscissa represents frequency and the axis of ordinates represents signal gain, shows the gain of signal frequency including transmission/reception in the case in which the entire band BND is assumed only at the surface of the probe without taking into consideration the dependence of distance and biological decay. The entire band BND is divided into subbands BD0 to BD3 from the lower frequency side and for each of them, beamformer processing is conducted separately.

FIG. 10(2), in which the axis of ordinates represents signal attenuation and the axis of abscissa represents frequency as with (1), conceptually shows signal attenuations ATT1, ATT2 at different depth ranges DP1 and DP2 (DP2>DP1) in the body. In general, as the depth increases, the signal attenuation (dB) of echo increases. Besides distance dependent attenuation due to diffusion, frequency dependent attenuation due to soundwave absorption and scattering of the living body is known and it is widely known that as the frequency increases, the attenuation increases. Since, for this reason, the degree of attenuation differs depending on frequency, upon compensating for these by modification of transmit waveform and dynamic gain adjustment during reception, compensation in accordance with frequency will be required. For this purpose, as with the example of FIG. 10(1), an improvement is achieved by dividing the entire band BND into a predetermined number of subbands BD0 to BD3 from the lower frequency side, and conducting separate beamformer processing respectively. That is, compensation is conducted separately for each band and the distribution of those compensation amounts is varied in time. Further, when compensating for changes of delay time due to the differences of sound velocity in living tissues and organs, if they are expected to depend on frequency, the delay time data will be provided by dividing it in frequency. As a result of this as well, an improvement will be achieved by conducting the processing of subbands BD0 to BD3 using separate beamformer data respectively.

FIG. 10(3) shows the case in which in the simultaneous time divided multiple access of four beams of FIG. 3(3), the simultaneous time divided multiple access with assignment of four-band processing is conducted in stead of the processing of each beam. It is possible to conduct the processing by replacing the beam number NBM with band numbers 0, 1, 2, and 3. In this way, it is possible to realize operation of beamformer depending on frequency by: performing beamformer data computation for the same receive signal by dividing it into multiple subbands in the same manner as with the beam number NBM; performing the compensation for the frequency dependent attenuation and the correction of the delay time due to the dispersion of sound velocity to the object to be inspected with frequency being the variable other than time; and by preparing the delay and weighing data optimized for separate frequency bands subjected to signal band division and performing time-division beam processing independently treating it like a different beam.

Next, description will be made on the case in which transmitting beamformer data is referenced from receiving beamformer data to be used in common. Since when performing the scanning in many different beam deflection directions, transmitting beamformer data increases, and the efficiency of adjustment will be improved by creating it by referencing the receiving beamformer data and using it after making modifications by the parameters. In FIG. 12, from the range RF with respect to transmit focus F_{F} on NC and the sound velocity Vc, concerned receiving time TA′ is calculated by

TA′=2RF/Vc+TA_{C}

using an appropriate constant TA_{C} By accumulating and comparing the values of TA′ and the values of respective time spans S1 to Sk, as with the example shown in FIGS. 6 and 7, it can be calculated that the concerned time span is S3 and the intra-span time position from the lead of the time span S3 is TTX. Such computation is set in the time divided multiple access sequencer SQGN via the bus PA by the processor TCPU of FIG. 1. The coefficient sequencer SQLD reads out coefficients A_{τ1}, A_{w1}, A_{τ2}, A_{w2}, B_{τ1} to B_{τn}, B_{w1} to B_{wn}, C_{τ1} to C_{τn}, C_{w1} to C_{wn}, initial constant terms D_{τ} and D_{w} from the storage means EXTRM. The initial constant terms D_{τ} and D_{w} utilize the final values of the accumulation up to the span S2. The time divided multiple access sequencer SQGN outputs Tₛ at a time position in S3 span in accordance with TTX. The output DAT is transferred to the transmitting circuit means. By the means of providing time-counter fixed time value Tₛ, it is possible to realize transmit data and receive data by the same computing means as well as to provide means of performing adjustment by giving a change dependent on the intra-span time T_{S} and the various parameters P₁ to Pn. For example, modification of focal distance for transmission, minute change of the steering direction angle (ψ,φ) of the transmit beam, minute displacement of the transmit aperture position, and the like can be generated by computation without storing separate transmit data for every transmitting focus into the storage means EXTRM.

### INDUSTRIAL APPLICABILITY

Application of the present invention to the transmitting/receiving beamformer (phase regulator circuit) of a medical ultrasound imaging system will enable equipment having high efficiency and adjustment flexibility to be realized.

1. A diagnostic ultrasound imaging system comprising an ultrasonic beamformer which provides a different delay time and weighting for response to a plurality of transmit and receive elements of ultrasound transducer and transmits/receives ultrasound into and from a living body, said diagnostic ultrasound imaging system characterized in that
said ultrasonic beamformer comprises numerical computation means which sequentially iterates processing to compute beamformer data made up of said delay time and weighting for response by using a function including a combination of linear terms of plural variables with quadratic term of a time variable within a partitioned time span.

2. The diagnostic ultrasound imaging system according to claim 1, characterized in that
said numerical computation means sequentially iterates the processing to compute beamformer data made up of said delay time and weighting for response by using a function including a combination of linear term of said plural variables, quadratic terms which are products of time variable and one of the plural variable, and said quadratic term of a time variable within a partitioned time span.

3. The diagnostic ultrasound imaging system according to claim 1, characterized in that
the time spans for the delay time and the weighting for response of said beamformer data are shared.

4. The diagnostic ultrasound imaging system according to claim 1, characterized in that
at least a computation processing order is arranged such that a constant term is added in the first time span, and in a subsequent time span, computation is performed to retain a last computed value of an immediately preceding time span as an initial value of said subsequent span.

5. The diagnostic ultrasound imaging system according to claim 1, characterized in that
a coefficient or an initial value included in said function is retained in storage means, and these are successively read out to be used for computation, from the storage means before the start of computation of each time span.

6. The diagnostic ultrasound imaging system according to claim 5, characterized in that
an operation mode is selectable in which read-out operation of coefficients excepting an initial value and a coefficient of a linear function of only time is omitted.

7. The diagnostic ultrasound imaging system according to claim 1, characterized in that
said time spans are made common for every predetermined number of times of channel time divided multiple access of said beamformer or every predetermined beam time divided multiple access condition.

8. The diagnostic ultrasound imaging system according to claim 1, characterized in that
the time span size is a common multiple of a predetermined number of times of channel time divided multiple access or the number of times of beam time divided multiple access.

9. The diagnostic ultrasound imaging system according to claim 4, characterized in that
at least one of an incremental value of the time variable or an initial value to be added to the time function computation of each span is changed for every predetermined number of channel processes or every predetermined beamforming condition in a time segment, and the same time variable value is used to perform the computation in the final computation of the span.

10. The diagnostic ultrasound imaging system according to claim 1, characterized in that
the time segment is used being segmented into unequal intervals.

11. The diagnostic ultrasound imaging system according to claim 1, characterized in that
at least one of a quadratic term and a linear term of time only, and a variable term of other than time is provided, and beamformer data is computed depending on the transmitting/receiving direction or transmitting/receiving aperture position of ultrasound.

12. The diagnostic ultrasound imaging system according to claim 1, characterized by comprising
computation means for determining the computation coefficients of a beamformer by a linear programming consisting of regularizing condition inequalities which are multiply divided into time spans.

13. The diagnostic ultrasound imaging system according to claim 7, characterized in that
at least part of beam time divided multiple access comprises providing computation coefficients respectively corresponding to frequency bands obtained by dividing a signal band.

14. The diagnostic ultrasound imaging system according to claim 1, characterized in that
disabling processing of the beamformer computation result by a time counter is further performed.

15. The diagnostic ultrasound imaging system according to claim 1, characterized in that
means for providing at least a time value of a time function is provided, and beamformer data computing means for transmission and reception are shared.

16. The diagnostic ultrasound imaging system according to claim 1, characterized in that
a change amount of a time variable in a time function in said time span is changed for every predetermined number of times of channel time divided multiple access or every predetermined beam time divided multiple access condition of said beamformer.

17. The diagnostic ultrasound imaging system according to claim 2, characterized in that
said multiple variables correspond to a function which designates the transmitting/receiving direction of said beamformer.

18. The diagnostic ultrasound imaging system according to claim 17, characterized in that
said multiple variables are partial differential coefficients of a function which designates the transmitting/receiving direction of said beamformer.

19. The diagnostic ultrasound imaging system according to claim 1, characterized in that
said ultrasound beamformer causes the time-wise 1st partial differential coefficients to substantially meet at a connection point in said time span.

20. The diagnostic ultrasound imaging system according to claim 1, characterized in that
said plurality of transmit and receive elements of ultrasound transducer make up a probe, and that
at least one element located in the vicinity of both ends of the aperture of said probe is assigned 0 as the weighting for response, and at least one element located in the vicinity of the center of said aperture is assigned weighting other than 0 as the weighting for response.
